(19) United States
(12) Patent Application Publication
(10) Pub. No.: US 2005/0004144 A1
(43) Pub. Date: Jan. 6, 2005

(54) Combined use of IMPDH inhibitors with toll-like receptor agonists

(75) Inventors: Dennis A. Carson, La Jolla, CA (US); Howard B. Cottam, Escondido, CA (US); Jongdae Lee, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 10/824,833

(22) Filed: Apr. 14, 2004

Related U.S. Application Data
(60) Provisional application No. 60/463,152, filed on Apr. 14, 2003.

(51) Int. Cl. A61K 31/519; C07D 498/02; C07D 487/02

(52) U.S. Cl. 514/260.1; 514/264.11; 514/265.1; 544/255; 544/279; 544/280

(57) ABSTRACT

The present invention provides a broad-spectrum, long-lasting, and non-toxic combination of synthetic immunostimulatory agents, which are useful for activating the immune system of a mammal and treating diseases such as cancer and autoimmune disease. These agents include TLR-ligands and ligand analogs which induce interferon production, in combination with inhibitors of inosine monophosphate dehydrogenase (IMPDH), that further enhance the induction of interferon production.

### CROSS-REFERENCES TO RELATED APPLICATIONS

This application claims the benefit of U.S. Provisional Application No. 60/463,152, filed Apr. 14, 2003; which is herein incorporated by reference for all purposes.

### BACKGROUND OF THE INVENTION

A great deal has been learned about the molecular basis of innate recognition of microbial pathogens in the last decade. It has been established that many somatic cells express a range of pattern recognition receptors that detect potential pathogens independently of the adaptive immune system (Janeway et al., Annu Rev Immunol, 20:197-216 (2002)). These receptors interact with microbial components termed pathogen associated molecular patterns (PAMPs). Examples of PAMPs include peptidoglycans, lipotechoic acids from gram-positive cell walls, the sugar mannose (which is common in microbial carbohydrates but rare in humans), bacterial DNA, double-stranded RNA from viruses, and glucans from fungal cell walls. By definition, PAMPs meet certain criteria that include, (a) their expression by microbes but not their mammalian hosts, (b) conservation of structure across the wide range of pathogens, and (c) the capacity to stimulate innate immunity. Recently, Toll-like Receptors (TLRs) have been found to play a central role in the detection of PAMPs and in the early response to microbial infections (Underhill et al., Curr Opin Immunol, 14:103-110 (2002)). Ten mammalian TLRs and a number of their ligands have been identified. For example, TLR7 and TLR9 recognize and respond to imiquimod and immunostimulatory CpG oligonucleotides (ISS-ODN), respectively. The synthetic immunomodulator R-848 (resiquimod) activates both TLR7 and TLR8. While TLR stimulation initiates a common signaling cascade (involving the adaptor protein MyD88, the transcription factor NF-kB, and pro-inflammatory and effector cytokines), certain cell types tend to produce certain TLRs. For example, TLR7 and TLR9 are found predominantly on the internal faces of endosomes in dendritic cells (DCs) and B lymphocytes (in humans; mouse macrophages express TLR7 and TLR9). TLR8, on the other hand, is found in human blood monocytes. (Hornung et al., J Immunol, 168:4531-4537 (2002)).

Interferons (INFs) are also involved in the efficient induction of an immune response, especially after viral infection (Brassard et al., J Leukoc Biol, 71:568-581 (2002)). However, many viruses produce a variety of proteins that block interferon production or action at various levels. Indeed, antagonism of interferon is apparently part and parcel of a general strategy to evade innate, as well as adaptive immunity (Levy et al., Cytokine Growth Factor Rev, 12:143-156 (2001)). While TLR ligands (TLR-L) may be sufficiently active for some methods of treatment, in some instances the microbial interferon antagonists could mitigate the adjuvant effects of synthetic TLR-L. Accordingly, there is a need for compounds that augment TLR-induced signal transduction.

New compositions that act as TLR ligands, alone or in combination with compounds that hinder viral or bacterial obstruction of interferon production, would represent a significant advance in the art. The present invention provides for these new TLR ligand compounds and compositions, as well as methods of using them.

### BRIEF SUMMARY OF THE INVENTION

The present invention provides a broad-spectrum, long-lasting, and non-toxic combination of synthetic immunostimulatory agents, which are useful for activating the immune system of a mammal, preferably a human.

The compounds of the invention have a structure according to Formula I: 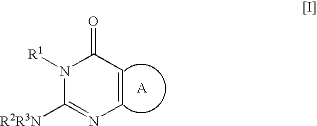
in which, R¹, R² and R³ represent members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. The ring system A is a member selected from Formula II: 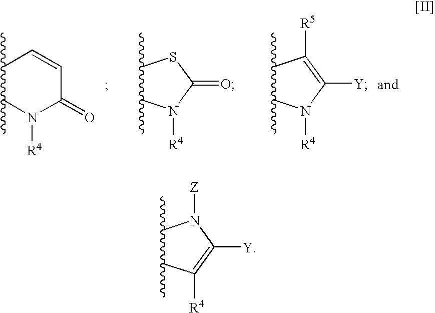
The symbol Z represents substituted or unsubstituted alkyl. Y is a member selected from H, halogen, nitro, and nitroso. The symbol R⁴ represents a member selected from H, substituted or unsubstituted alkyl, and substituted or unsubstituted heteroalkyl. R⁵ is a member selected from H, CN, OR¹², C(X¹)OR¹², C(X¹)NR¹³R¹⁴, NR¹⁵R¹⁶, SR¹², NO, halogen, substituted or unsubstituted C₁-C₆ alkyl and substituted or unsubstituted C₁-C₆heteroalkyl. R¹² is a member selected from H, substituted or unsubstituted C₁-C₆ alkyl, substituted or unsubstituted C₁-C₆ heteroalkyl and C(O)R¹⁷. The symbol R¹⁷ represents substituted or unsubstituted C₁-C₆ alkyl and substituted or unsubstituted C₁-C₆ heteroalkyl. X¹ is a member selected from (═O), (═NH) and (═S). The symbols R¹³ and R¹⁴ represent members independently selected from H, substituted or unsubstituted C₁-C₆ alkyl and substituted or unsubstituted C₁-C₆ heteroalkyl. R¹⁵ and R¹⁶ are members independently selected from H, O, substituted or unsubstituted C₁-C₆ alkyl and substituted or unsubstituted C₁-C₆ heteroalkyl, or, when taken together, form C(O)R⁸. The symbol R¹⁸ is a member selected from substituted or unsubstituted C₁-C₆ alkyl and substituted or unsubstituted C₁-C₆ heteroalkyl.

In another aspect, the present invention is a nucleic acid having a sequence comprising at least one moiety having a structure of formula I wherein the ring system A is a member selected from Formula V: 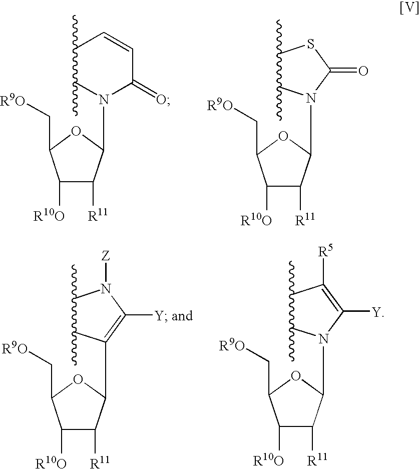
R⁹ and R¹⁰ are members independently selected from H and a nucleic acid. The symbol R¹¹ is a member selected from H, OH, and a nucleic acid.

In another aspect, the present invention provides pharmaceutical compositions comprising a pharmaceutically acceptable excipient and a compound of Formula I possessing a ring system according to Formula II. In another aspect, the present invention provides pharmaceutical compositions comprising a pharmaceutically acceptable excipient and a compound of Formula I possessing a ring system according to Formula V.

The present invention provides a method of activating an immune system in a mammal by administering a therapeutically effective amount of pharmaceutical composition containing a nucleic acid as described above, wherein the nucleic acid comprises a toll-like receptor (TLR) ligand. In one embodiment, the TLR ligand binds to a TLR expressed on an endosomal membrane. In additional embodiments a CpG oligonucleotide (ISS-ODN) or an IMPDH inhibitor is also administered. In a further embodiment, the composition is administered to a mucus membrane. In one aspect, the TLR ligand can be a homofunctional TLR ligand polymer and can consist of a TLR-7 ligand or a TLR-8 ligand. The TLR7 ligand can be a 7-thia-8-oxoguanosinyl (TOG) moiety, a 7-deazaguanosinyl (7DG) moiety, a resiquimod moiety, or an imiquimod moiety. The TLR8 ligand can be a resiquimod moiety. In another aspect, the TLR ligand is a heterofunctional TLR ligand polymer. The heterofunctional TLR ligand polymer can include a TLR-7 ligand and a TLR-8 ligand or a TLR-9 ligand or all three ligands. The heterofunctional TLR ligand polymer can include a TLR-8 ligand and a TLR-9 ligand.

The present invention provides method of enhancing resistance to infection in a mammal by administering a therapeutically effective amount of pharmaceutical composition containing a nucleic acid as described above, wherein the nucleic acid comprises a toll-like receptor (TLR) ligand. In one embodiment, the TLR ligand binds to a TLR expressed on an endosomal membrane. In additional embodiments a CpG oligonucleotide (ISS-ODN) or an IMPDH inhibitor is also administered. In a further embodiment, the composition is administered to a mucus membrane. In one aspect, the TLR ligand can be a homofunctional TLR ligand polymer and can consist of a TLR-7 ligand or a TLR-8 ligand. The TLR7 ligand can be a 7-thia-8-oxoguanosinyl (TOG) moiety, a 7-deazaguanosinyl (7DG) moiety, a resiquimod moiety, or an imiquimod moiety. The TLR8 ligand can be a resiquimod moiety. In another aspect, the TLR ligand is a heterofunctional TLR ligand polymer. The heterofunctional TLR ligand polymer can include a TLR-7 ligand and a TLR-8 ligand or a TLR-9 ligand or all three ligands. The heterofunctional TLR ligand polymer can include a TLR-8 ligand and a TLR-9 ligand. The present can enhance resistance to a viral infection, and in a preferred embodiment enhances resistance to an interferon sensitive virus. The present invention can also enhance resistance to a bacterial infection, and in a preferred embodiment enhances resistance to an intracellular bacterial infection. In further embodiments, an antibiotic is also administered to enhance resistance to a bacterial infection.

The present invention also provides a method of treating a viral infection in a mammal by administering a TLR ligand in combination with an IMPDH inhibitor. The TLR ligand can be a synthetic TLR ligand. In one embodiment, the IMPDH inhibitor is mizoribine, an enantiomer of mizoribine, mizoribine base, mizoribine aglycone, or a prodrug of such compound. In another embodiment, the viral infection is caused by an RNA virus and the RNA virus or a product of the RNA virus acts as a TLR ligand. Preferred RNA virus the coronavirus that causes Severe Acute Respiratory Syndrome (SARS) and the Hepatitis C Virus. In one aspect, the RNA virus is mutated and does not cause an induction of interferon synthesis without intervention. The IMPDH inhibitor can then be administered directly to the site of viral infection, for example, in the case of a coronavirus that causes SARS and the IMPDH inhibitor is administered to the lung, by inhalation. TLR ligands can be administered in combination with IMPDH inhibitors to treat a viral infection caused by a DNA virus. For treatment of a DNA virus, a preferred administration is systemic administration of the IMPDH inhibitor. A preferred DNA virus for treatment is the Hepatitis B virus.

The present invention provides a method for treating cancer by administering an inhibitor of inosine monophosphate dehydrogenase (IMPDH) and an interferon inducer, for example, a TLR ligand. The TLR ligand can be a component of a nucleic acid. In a preferred embodiment, the cancer is an interferon sensitive cancer, for example, a leukemia, a lymphoma, a myeloma, a melanoma, or a renal cancer. In another preferred embodiment the IMPDH inhibitor is mizoribine, mizoribine base, or mizoribine aglycone. The cancer treatment can also include a therapeutically effective amount of a Type I interferon. In one embodiment, the TLR ligand binds to a TLR expressed on an endosomal membrane. In additional embodiments a CpG oligonucleotide (ISS-ODN) or an IMPDH inhibitor is also administered. In a further embodiment, the composition is administered to a mucus membrane. In one aspect, the TLR ligand can be a homofunctional TLR ligand polymer and can consist of a TLR-7 ligand or a TLR-8 ligand. The TLR7 ligand can be a 7-thia-8-oxoguanosinyl (TOG) moiety, a 7-deazaguanosinyl (7DG) moiety, a resiquimod moiety, or an imiquimod moiety. The TLR8 ligand can be a resiquimod moiety. In another aspect, the TLR ligand is a heterofunctional TLR ligand polymer. The heterofunctional TLR ligand polymer can include a TLR-7 ligand and a TLR-8 ligand or a TLR-9 ligand or all three ligands. The heterofunctional TLR ligand polymer can include a TLR-8 ligand and a TLR-9 ligand.

The present invention provides a method for treating an autoimmune disease by administering a therapeutically effective amount of an inhibitor of inosine monophosphate dehydrogenase (IMPDH) and an interferon inducer. The interferon inducer can be a nucleic acid comprising a TLR ligand. In a preferred embodiment, the IMPDH inhibitor is mizoribine, mizoribine base, or mizoribine aglycone. In another preferred embodiment, the autoimmune disease is multiple sclerosis. In a further embodiment, a therapeutically effective amount of a Type I interferon is administered with the interferon inducer and IMPDH inhibitor. In one embodiment, the TLR ligand binds to a TLR expressed on an endosomal membrane. In additional embodiments a CpG oligonucleotide (ISS-ODN) or an IMPDH inhibitor is also administered. In a further embodiment, the composition is administered to a mucus membrane. In one aspect, the TLR ligand can be a homofunctional TLR ligand polymer and can consist of a TLR-7 ligand or a TLR-8 ligand. The TLR7 ligand can be a 7-thia-8-oxoguanosinyl (TOG) moiety, a 7-deazaguanosinyl (7DG) moiety, a resiquimod moiety, or an imiquimod moiety. The TLR8 ligand can be a resiquimod moiety. In another aspect, the TLR ligand is a heterofunctional TLR ligand polymer. The heterofunctional TLR ligand polymer can include a TLR-7 ligand and a TLR-8 ligand or a TLR-9 ligand or all three ligands. The heterofunctional TLR ligand polymer can include a TLR-8 ligand and a TLR-9 ligand.

The present invention provides a method of treating a disease accessible to topical treatment by topically administering an interferon inducer directly to the diseased tissue, and also administering a therapeutically effective amount of an inhibitor of inosine monophosphate dehydrogenase (IMPDH). The interferon inducer can be a nucleic acid comprising a TLR ligand, including ISS-ODN, or a monomeric TLR ligand, such as resiquimod, imiquimod, or other guanosine congener. In a preferred embodiment, the IMPDH inhibitor is administered systemically. In another preferred embodiment, the IMPDH inhibitor is a member selected from the group consisting of mizoribine, mizoribine base, and mizoribine aglycone. In one aspect the disease accessible to topical treatment is a cancer, such as a melanoma, superficial bladder cancer, actinic keratoses, intraepithelial neoplasia, or basal cell skin carcinoma. In one aspect the disease accessible to topical treatment is a precancerous condition, such as actinic keratoses and intraepithelial neoplasia. In a further aspect, the disease accessible to topical treatment is a viral disease, such as a human papilloma virus infection, a molluscum contagiosum, or a herpes virus infection.

The present invention provides a method of treating cancer by administering a therapeutically effective amount of a member selected from mizoribine, mizoribine base, mizoribine aglycone, an enantiomer of such a compound, a prodrug of such a compound, a pharmaceutically acceptable salt of such a compound, and combinations thereof; in combination with a therapeutically effective amount of Type I interferon. In one embodiment, the cancer is a leukemia, a lymphoma, a myeloma, a melanoma, or a renal cancer.

The present invention provides a method of treating a viral infection by administering a therapeutically effective amount of a member selected from mizoribine, mizoribine base, mizoribine aglycone, an enantiomer of such a compound, a prodrug of such a compound, a pharmaceutically acceptable salt of such a compound, and combinations thereof; in combination with a therapeutically effective amount of Type I interferon. In one embodiment the viral infection is caused by a coronavirus that causes Severe Acute Respiratory Syndrome (SARS), a Hepatitis B virus, or a Hepatitis C Virus.

The present invention provides a method of treating an autoimmune disease by administering a therapeutically effective amount of a member selected from mizoribine, mizoribine base, mizoribine aglycone, an enantiomer of such a compound, a prodrug of such a compound, a pharmaceutically acceptable salt of such a compound, and combinations thereof; in combination with a therapeutically effective amount of Type I interferon. In one embodiment, the autoimmune disease is Multiple Sclerosis.

The present invention provides a method of treating Crohn's Disease by administering an inhibitor of inosine monophosphate dehydrogenase (IMPDH), and a probiotic or glycolipid.

These and other objects, aspects and embodiments of the invention will be apparent from the detailed description that follows.

### BRIEF DESCRIPTION OF THE DRAWINGS

FIG. 1 depicts the chemical structures of guanosine; the guanosine inhibitors 7-deazoguanosine and 7-thia-8-oxoguanosine (TOG); and the IMPDH inhibitors ribavirin, misoribine, and mizoribine base.

FIG. 2 depicts antigen-induced splenocyte cytokine profiles. Female BALB/c mice received three immunizations with β-gal (50 ug) alone or with ISS-ODN (50 μg) 7 days apart, via the intranasal or id route. Splenocytes were harvested from sacrificed mice during week 7 and cultured in medium with or without β-gal (10 ug/ml), and ELISAs were assayed on 72-h supernatants. Splenocytes from immunized mice cultured without β-gal produced negligible amounts of cytokines (data not shown). Results represent the mean of four mice in each group and similar results were obtained in two other independent experiments. Error bars reflect standard errors of the means (A) IFNγ. (B) IL-6.

FIG. 3 depicts splenocyte CTL and IFN-γ response after intradermal ISS pre-priming. Results represent the mean ± SE for four mice in each group and similar results were obtained in two other independent experiments. Mice immunized with M-ODN either prior to or with β-gal immunization did not demonstrate an increased IFN-γ or CTL response when compared to mice immunized with β-gal along (data not shown). (a) IFN-γ response. Similar findings were observed for murine (i.n.) pre-priming (R). Mice receiving ISS up to 14 days prior to β-gal demonstrated an improved IFN-γ response when compared to mice immunized with β-gal alone (†P≦0.05). Delivery of ISS from 3-7 days before β-gal led to an increased IFN-γ response when compared to mice receiving ISS/β-gal co-immunization (*P≦0.05) (B) CTL response. (C) Comparison of CTL response at an effector:target ratio of 25:1. Mice receiving ISS up to 14 days prior to β-gal demonstrated an improved CTL response when compared to mice immunized with β-gal alone (†P≦0.05).

FIG. 4 depicts ISS inhibition of RSV replication in the lung. Panel A demonstrates that ISS inhibits the number of RSV plaque-forming units (log 10 scale) in lungs of mice infected with RSV and treated with ISS compared with that seen in mice infected with RSV and treated with M-ODN (n=3; *P<0.001). Similarly, Panel B demonstrates, by means of RT-PCR, that ISS inhibits the level of expression of the RSV-N gene in the lungs of mice infected with RSV and treated with ISS compared with that seen in mice infected with RSV and treated with M-ODN. Control housekeeping gene L32 expression is also depicted.

FIG. 5 depicts the effect of ISS on RSV-induced peribronchial inflammation. RSV infection induced the expression of significant numbers of peribronchial inflammatory cells compared with that seen in uninfected mice (n=3; **P<0.05). ISS significantly inhibited the number of peribronchial inflammatory cells in the airways of RSV-infected mice treated with ISS compared with RSV-infected mice that had not received ISS (n=3; *P<0.05).

FIG. 6 depicts the effect of ISS on RSV induced BAL inflammation. RSV infection induced a significant increase in BAL lymphocytes compared with that seen in uninfected mice (n=3; *P<0.05). ISS inhibited the RSV induced increase in BAL lymphocytes compared with that seen in RSV-infected mice that had not received ISS, but this did not reach statistical significance (n=3; P=0.07).

FIG. 7 depicts the effects of TLR ligands on Listeria infection in mice.

FIG. 8 depicts the antimycobacterial effects of ISS-ODN in vitro. (A) mBMDMs were treated with ISS-ODN or M-ODN for 3 days prior to M-avium infection, and intracellular growth of M-avium was assessed by the CFU assay on days 1, 3, and 7 after infection. (B) mBMDMs were treated with ISS-ODN or M-ODN immediately after infection, and M-avium growth was assessed by the CFU assay 7 days post infection. Each condition was tested in triplicate, and the results are expressed as means ± SD CFU per well. The results shown are representative of three experiments.

FIG. 9 depicts activation of TLR7 by guanosine analogs. A. TOG activated TLR7. 293-HEK cells were transfected with the indicated human TLR with NF-kB reporter construct. On the next day, cells were treated with or without TOG (200 μM) for 6 hrs and TOG-induced luciferase activity was measured as readout of NF-kB activation. B. TOG does not activated human TLR8. 293-HEK cells were transfected with pCMV vector, TLR7, or TLR8, and treated with TOG (200 μM), Imiquimod (50 μM), or R-848 (20 μM) for 6 hrs before luciferase activity was measured. C. Guanosine analogs activated TLR7. 293-HEK cells transfected with TLR7 or TLR8 were treated with guanosine analogs (200 μM), or Imiquimod, R-848 and luciferase activity was measured as above. D. Guanosine analogs induce Type I IFNs in human peripheral blood leukocytes. Mononuclear cells, isolated with a ficoll gradient, were treated with TLR ligands (guanosine analogs: 100 μM, R-848: 1 μM, LPS: 10 ng, CpG: 10 μg) for the indicated time periods, and total RNA was used for RT-PCR. All the stimuli were able to induce IFNα and IFNβ.

FIG. 10 depicts an immunostimulatory ODN (100 μM) in water was injected in 50 μl volume onto a size exclusion TSK-Gel G2000SW_{XL} HPLC column. Elution was carried out in buffer containing 10 mM sodium phosphate, pH 6.9, 0.3 M NaCl at a flow rate of 0.6 ml/min. Fractions were collected beginning at 9.5 min up to 12.5 min for high molecular weight aggregates, followed by collection of monomer ODNs and ended at 15 min. ODN concentrations were determined by absorption at 260 nm and then sterilized. Afterwards, equal amounts and volumes of either aggregated or monomeric ODN fractions were added to mouse bone marrow-derived macrophages and stimulated for 48 hours. Media were collected and IL-12p40 production was analyzed by ELISA. A 9.75-fold increase in IL-12 production was observed from cells stimulated with aggregated ODN compared to that with equal amounts of the monomer.

FIG. 11 depicts IMPDH inhibitors enhance TLR signaling through activation of IRFs (interferon regulatory factors). A. Ribavirin enhances TLR-mediated production of IL-12 (p40) in a dose dependent manner. Mouse bone marrow-derived macrophages (BMDM) were stimulated with TLR ligands (TOG:100 μM, R-848:1 μM, Pam3Cys:5 μg, p(I:C):5 μg, LPS:10 ng, ISS:5 μg) for 24 hrs in the absence or presence of ribavirin (10, 50, 100 μM) and IL- 12 production was measured by ELISA. Ribavirin alone did not induce any cytokines. B. Mizoribine or mizoribine base synergize with TOG in a similar manner. BMDM were treated with TOG (100 μM) in the absence or presence of mizoribine (10 μM), or mizoribine base (10 μM), and IL-12 production was measured after 24 hrs. C. IMPDH inhibitors induce activation of IRF-3 and IRF-7 but not NF-kB or STAT-1. BMDM were treated with ribavirin (R), mizoribine (M), mizoribine base (Mb), or TOG for 4 hrs, and activation of IRF-3 and IRF-7 was measured by immunoblotting in nuclear extracts. Phosphorylated IRF-3 and IRF-7 are translocated to nucleus. Activation of STAT-1 was also measured with phospho-specific antibody against STAT-1. NF-kB activation was measured by EMSA. Only TOG was able to activate NF-kB and STAT-1, indicating that IMPDH inhibitor alone does not induce Type I IFNs in these cells. D. IMPDH inhibitors enhance TOG-induced activation of NF-kB and IRF-1. BMDM were treated with or without IMPDH inhibitors and activation of NF-kB (EMSA) or IRF-1 (immunoblotting) was measured in nuclear extracts. Ribavirin or mizoribine alone did not induce activation of NF-kB or IRF-1 but strongly enhanced TOG-induced activation of both molecules.

FIG. 12 demonstrates that activation of IRFs by IMPDH Inhibitors is dependent on DNA-dependent protein kinase (DNA-PK). Activation by mizoribine base (Mb) is shown. Experiments were performed in cells from wild-type mice and SCID mice, which lack functional DNA-PK.

FIG. 13 demonstrates that activation of IRFs by TLR7 is via DNA-PK But not MyD88.

FIG. 14 demonstrates that activation of IRFs by TLR7 and Mb is via DNA-PKcs but not MyD88.

FIG. 15 demonstrates that augmentation of TLR-mediated cytokine induction by IMPDH inhibitors is DNA-PKcs dependent. The experiment was performed in cells from SCID mice.

FIG. 16 demostrates that mizoribine base (Mb) augments TLR-7 mediated type I interferon production both in spenocytes and in vivo. In the left panel, splenocytes were stimulated with TOG or R-8484 with or without Mb (10 μM) for 24 hrs and type I interferon was measured by bioassay. In the right panel, B57/b6 mice were injected intravenously with TOG (250 μg) and increasing doses of Mb. Type I interferon in serum was measured and results are shown.

FIG. 17 demonstrates that IMPDH inhibitors enhance TLR7-induced IL-12 production in human peripheral blood leukocytes (hPBL). In the left panel, hPBL were stimulated with TOG (100 μM) in the presence or absence of Rb, Mb or MPA for 24 hours. IL-12 was then measured by ELISA and results are depicted. In the right panel, hPBL were stimulated with R-848 (1 μM) plus increasing doses of Mb or ribavirin (Rb) for 3 hrs and activation of STAT-1 was measured by western blotting. β-actin levels were measured as a control

FIG. 18 demonstrates that IMPDH inhibition augments TLR-7 mediated activation in bone marrow derived macrophages or in bone marrow derived dendritic cells (DC's). The left panel shows results in BMDM. Rb enhances TOG-induced NF-κB activation and activates IRF-3 transcription factor. BMDM were stimulated with TOG with or without Rb (50 μM) or Mb (10 μM) for the indicated period and NF-κB activation was measured by electrophorectic mobility shift assay (EMSA) and IRF-3 by western blotting. The right panel shows results in dendritic cells. Mb enhanced TLR7 signaling in DC's. Bone marrow derived DC's were stimulated by R-848 with or without Mb for the indicated time period, and activation of NF-κB, MAP kinases (p38 and EKR) was measured. Actin levels were measured as a control.

FIG. 19 demonstrates activation of DNA dependent protein kinase catalytic subunit (DNA-PKcs) by IMPDH inhibitors and TOG. IMPDH inhibitors and TLR7 activate DNA-PK. BMDM were stimulated with the Mb alone, TOG alone, or a combination of Mb and TOG. Activation of DNA-PK was measured by in vitro kinase assay using a GST-p53 substrate or DNA-PKcs autophosphorylation.

FIG. 20 demonstrates that induction of type I interferon by TOG combined with Mb is partially dependent on DNA-PKcs. Splenocytes of WT or SCID mice were stimulated with the indicated stimuli and Type I interferon was by bioassay. TOG (100 μM), R-848 (1 μM), and Mb (10 μM).

FIG. 21 demonstrates that activation of IKKi/ε is partially dependent on DNA-PKcs. BMDM of WT or SCID mice were stimulated with the indicated stimuli and activation of IKKi/ε was measured by in vitro kinase assay.

### DETAILED DESCRIPTION OF THE INVENTION

Introduction

The present invention provides a broad-spectrum, long-lasting, and non-toxic combination of synthetic immunostimulatory agents. Also provided are compounds that are composed of at least one homofunctional or heterofunctional TLR ligand or TLR ligand analog. These compounds can include cross-linked synthetic ligands for a single TLR or for multiple TLRs.

The compounds of the invention can be used to enhance resistance to infection in a mammal and/or to treat disease. Exemplary diseases include cancer and autoimmune disease. The TLR ligands of the invention induce cellular synthesis of interferon. The compounds can also be given in combination with IMPDH inhibitors, which enhance interferon production induced by TLR ligands, thus further enhancing an immune system response, and in some embodiments providing a synergistic treatment of a condition of interest.

Abbreviations and Definitions

The abbreviations used herein have their conventional meaning within the chemical and biological arts.

A “method of activating an immune system in a mammal” refers to stimulation of an immune system component, usually by stimulating a toll-like receptor. The innate immune system, the adaptive immune system, or both can be activated.

A “toll-like receptor” (TLR) refers to a member of a family of receptors that bind to pathogen associated molecular patterns (PAMPs) and facilitate an immune response in a mammal. Ten mammalian TLRs are known, e.g., TLR1-10.

A “toll-like receptor ligand” (TLR ligand) refers to a molecule that binds to a TLR and activates the receptor. A TLR ligand can be naturally occurring, e.g. PAMPs. Synthetic TLR ligands are chemical compounds that are designed to bind to a TLR and activate the receptor. Exemplary novel TLR ligands provided herein include “TLR-7 ligand” “TLR-8 ligand” and “TLR-9 ligand.”

A “toll-like receptor expressed on an endosomal membrane” refers to a TLR that is localized to an endosome and binds to TLR ligands that have been internalized through endocytosis.

A “mucus membrane” refers to a membrane that lines openings or canals of the body that open to the outside. Examples include the linings of the mouth, digestive tube, breathing passages, and the genital and urinary tracts. Mucous membranes release mucus, and absorb water, salts, and other substances. Exemplary mucus membranes include membranes of the conjunctiva, nasopharynx, oropharynx, vagina, urethra, and urinary bladder.

“Essentially non-antigenic” describes a molecule that does not elicit an immune response against itself when administered to a mammal.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 7-position purine modifications, 8-position purine modifications, 9-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a BHQ, a fluorophore or another moiety.

A “homofunctional TLR ligand polymer” refers to a molecule comprising more than one TLR ligand that are covalently bound and that bind to the same TLR. The TLR ligands that make up the polymer can be different molecules, so long as they all bind to the same TLR.

An “IMPDH inhibitor” refers to an inhibitor of the enzyme inosine monophosphate dehydrogenase. Currently, three IMPDH inhibitors are used clinically: ribavirin, mizoribine, and mycophenolate mofetil. Ribavirin and mizoribine are prodrugs that are phosphorylated intracellularly to produce IMP analogs (Goldstein et al., Cuff Med Chem, 6:519-536 (1999)). Viramidine is a prodrug of Ribavirin. Mycophenolate mofetil is immunosuppressive, and has gastrointestinal irritative properties that may be attributable to its enterohepatic recirculation (Papageorgiou C, Mini Rev Med Chem., 1:71-77 (2001)). Mizoribine aglycone, a prodrug, is used as an IMPDH inhibitor. Other IMPDH inhibitors are Other IMPDH inhibitors, including prodrugs of mizoribine and mizoribine aglycone are known and are described in U.S. Patent Application Nos. 60/400,583 and 60/400,568, both filed Aug. 2, 2002 and both of which are herein incorporated by reference.

Other IMPDH inhibitors include Tiazofurin. Tiazofurin is anabolized to become an NAD analog that inhibits IMPDH. Tiazofurin may be prepared as described in U.S. Pat. No. 4,680,285 or U.S. Pat. No. 4,451,648, incorporated herein by reference. Selenazofurin, benzamide riboside, 6-CL-IMP, and VX-497 are also IMPDH inhibitors.

A “heterofunctional TLR ligand polymer” refers to a molecule comprising more than one TLR ligand that binds to different TLRs. For example, a heterofunctional TLR ligand polymer can be made up of TLR-7 and TLR-9 ligands covalently bound or otherwise associated e.g. electrostatic interactions.

A “method of enhancing resistance to infection in a mammal” includes methods to enhance resistance to bacterial infection, i.e., caused by a bacteria, and methods to enhance resistance to viral infection, i.e., caused by a virus. The term includes decreasing the likelihood of a subject becoming infected by a bacteria or virus and, if infection occurs, shortening the duration of the infectious illness. In some preferred embodiments, the methods can be used to treat interferon-sensitive virus. In other preferred embodiments, the methods can be used to treat an intracellular bacterial infection. Examples of bacteria that cause intracellular bacterial infections include Mycobacterium tuberculosis, Mycobacterium bovis, Mycobacterium leprae, Salmonella enterica, Brucella, Legionella pneumopholia, Listeria moncyotgenes, Francisella tularensis, Rickettsia rickettsii, Rickettsia prowazeki, Rickettsia typhi, Rickettsiatsutsujamushi, Coxiella burnetii, Chlamydia trachomatis, Chlamydia psittaci, Chlamydia pneumoniae, Shigella, Yersinia, and Toxoplasma gondii.

A “method for treating cancer” refers to a method of eliminating or decreasing the number of cancer cells in a mammal. Anticancer effects can result from a direct effect on cancer cells, such as inhibition or decrease in rate of proliferation or induction of apoptosis; or from an indirect effect on immune effector populations that interact with tumor cells; or from inhibition of angiogenesis.

An “interferon inducer” refers to a compound that induces cellular interferon expression. TLR ligands, both naturally occurring (e.g., either virus or bacteria or compounds produced by virus or bacteria) and synthetic, are interferon inducers. Other synthetic inducers of interferons are known and include double-stranded polynucleotides, tilorone, halopyrimidines, acridines, substituted quinolones, and flavone acetic acid.

An “interferon-sensitive cancer” refers to cancer that is amenable to treatment with interferon and includes leukemia, melanoma, renal cell cancer, myeloma, lymphoma, follicular cancer, T-cell cancer, multiple myeloma, midgut carcinoids, Kaposi's sarcoma, ovarian, basal cell, bladder, and breast cancer.

A “method for treating an autoimmune” refers to the reduction or elimination of the symptoms an autoimmune disease. Autoimmune diseases include diabetes, rheumatoid arthritis, multiple sclerosis, lupus erythematosis, myasthenia gravis, scleroderma, Crohn's disease, ulcerative colitis, Hashimoto's disease, Graves' disease, Sjogren's syndrome, polyendocrine failure, vitiligo, peripheral neuropathy, graft-versus-host disease, autoimmune polyglandular syndrome type I, acute glomerulonephritis, Addison's disease, adult-onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barre syndrome, hemochromatosis, Henoch-Schonlein purpura, idiopathic IgA nephropathy, insulin-dependent diabetes mellitus (IDDM), juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome and thyroiditis.

Multiple sclerosis (MS) is an exemplary autoimmune disease. Symptoms of MS, e.g., loss of vision, double vision, dizziness, weakness, loss of sensation, problems controlling bladder and bowel function, muscle weakness in their extremities and difficulty with coordination and balance, paresthesias, transitory abnormal sensory feeling such as numbness or “pins and needles,” pain, and cognitive impairments such as difficulties with concentration, attention, memory, and judgment. MS is an autoimmune disease and the activation of Th1 type T-cells is thought to be a primary component of the autoimmune response. In MS, the autoimmune response attacks the myelin sheath neuronal axons.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups which are limited to hydrocarbon groups are termed “homoalkyl”.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Thus, a cycloalkyl or heterocycloalkyl include saturated and unsaturated ring linkages. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo” as used herein means an oxygen that is double bonded to a carbon atom.

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R′″ are preferably independently selected from hydrogen, alkyl, heteroalkyl, aryl and heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula —T—C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)S—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S) and silicon (Si).

As used herein, “carrier moiety”, refers to species that selectively localize in a particular tissue or region of the body. The localization is mediated by specific recognition of molecular determinants, molecular size of the carrier moiety, ionic interactions, hydrophobic interactions and the like. Examples of these terms are cell-surface receptor ligands and antibodies, as well as liposomes and polymers that extend bioavailability prior to entry into the reticulo-endothelial system (RES). Other mechanisms of targeting a carrier moiety to a particular tissue or region are known to those of skill in the art.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds which are in a prodrug form. Prodrugs of the compounds described herein are those compounds that readily undergo chemical changes in vivo to provide the compounds of the present invention. For example, nucleosides can be esterified to increase uptake from the gut. The ester groups are then cleaved by enzymes in the body to yield the active product. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “ring” as used herein means an encircling arrangement of atoms optionally having heteroatoms within the arrangement. A ring includes aromatic and non-aromatic moieties such as substituted or unsubstituted cycloalkyl, substituted or unsubstituted heterocycloalkyl, substituted or unsubstituted aryl and substituted or unsubstituted heteroaryl.

The term “polymer” refers to any of numerous natural and synthetic compounds, of usually high molecular weight, consisting of repeated linked units.

The symbol [symbol] , whether utilized as a bond or displayed perpendicular to a bond indicates the point at which the displayed moiety is attached to the remainder of the molecule.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

Synthetic TLR Ligands

In a first aspect, the invention provides for administration of a polymer or nucleic acid comprising TLR7 or TL8 ligands. In another aspect the invention provides for administration of a polymer comprising a TLR7 ligand and a TLR8 or TLR9 ligand. Alternatively, the invention provide for co-administration of a TLR7 ligand and TLR8 ligand and a TLR9 ligand. The ligands can be administered as separate species, e.g. in a “cocktail”. Alternatively, combining two or more ligands to form a divalent or polyvalent drug, e.g. an ODN drug or prodrug, may yield immunostimulatory molecules with equivalent effects in both rodent and human systems.

Since nearly all human pathogens already express PAMPs capable of activating one or more TLR receptors, one can legitimately suppose that synthetic TLR ligands would be of minimal benefit in augmenting host resistance to infection. However, two phenomena argue strongly against this contention. Many intracellular pathogens also produce inhibitory molecules that can mitigate the effects of TLR signal transduction (Levy et al., Cytokine Growth Factor Rev, 12:143-156 (2001)). Moreover, the mucosal immune cells of normal people often display a tolerogenic phenotype when exposed as adults to exogenous antigens, possibly consequent to reduced stimulation by PAMPs during early immune development (Neurath et al., Nat Med, 8:567-573 (2002)). Thus, immune cells in the respiratory and gastrointestinal tracts of normal adults may not mount optimal immune responses to dangerous pathogens. In this situation, synthetic TLR ligands, together with pharmacologic potentiators of TLR signal transduction, are of use to correct the immune deficit.

Description of the Compounds

In a first aspect, the present invention provides compounds according to Formula I: 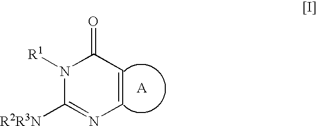
in which, R¹, R² and R³ represent members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. The ring system A is a member selected from Formula II: 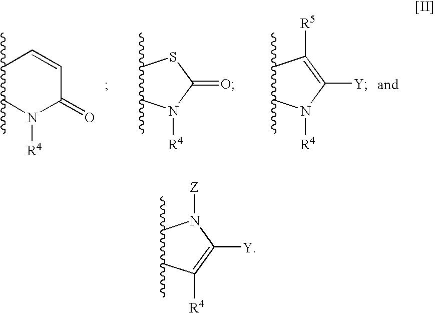
The symbol Z represents substituted or unsubstituted alkyl. Y is a member selected from H, halogen, nitro, and nitroso. The symbol R⁴ represents a member selected from H, substituted or unsubstituted alkyl, substituted or unsubstituted heteroalkyl. R⁵ is a member selected from H, CN, OR¹², C(X¹)OR¹², C(X¹)NR¹³R¹⁴, NR¹⁵R¹⁶, SR¹², NO, halogen, substituted or unsubstituted C₁-C₆ alkyl and substituted or unsubstituted C₁-C₆ heteroalkyl. R¹² is a member selected from H, substituted or unsubstituted C₁-C₆ alkyl, substituted or unsubstituted C₁-C₆ heteroalkyl and C(O)R¹⁷. The symbol R¹⁷ represents substituted or unsubstituted C₁-C₆ alkyl and substituted or unsubstituted C₁-C₆ heteroalkyl. X¹ is a member selected from (═O), (═NH) and (═S). The symbols R¹³ and R¹⁴ represent members independently selected from H, substituted or unsubstituted C₁-C₆ alkyl and substituted or unsubstituted C₁-C₆ heteroalkyl. R¹⁵ and R¹⁶ are members independently selected from H, O, substituted or unsubstituted C₁-C₆ alkyl and substituted or unsubstituted C₁-C₆ heteroalkyl, or taken together, form C(O)R¹⁸. The symbol R¹⁸ is a member selected from substituted or unsubstituted C₁-C₆ alkyl and substituted or unsubstituted C₁-C₆ heteroalkyl.

In an exemplary embodiment, R⁴ is a member selected from alkyl substituted with at least one hydroxyl moiety and heteroalkyl substituted with at least one hydroxyl moiety.

In another exemplary embodiment R⁴ has the structure according to Formula III: 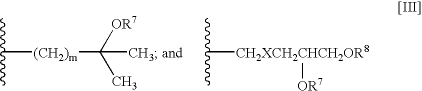
in which, m is an integer from 1 to 10. R⁷ and R⁸ are members independently selected from H and carrier moieties. The symbol X represents a member selected from O, S and NR⁶. R⁶ is a member selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

In an exemplary embodiment the carrier moiety is a polymer. In another exemplary embodiment the carrier moiety is essentially non-antigenic in a mammalian subject. In yet another exemplary embodiment the carrier moiety is a biomolecule. In another exemplary embodiment the carrier moiety is a member selected from a nucleic acid, an amino acid, a peptide, a peptide-amino acid, a saccharide, an antibody, an antigen, a lectin and combinations thereof.

In an exemplary embodiment R⁴ is a saccharyl moiety. In another exemplary embodiment, the saccharyl moiety is a member selected from substituted or unsubstituted ribofuranose and substituted or unsubstituted deoxyribofuranose. In yet another exemplary embodiment, the saccharyl moiety is part of a complex, said complex comprising a member selected from a nucleic acid and a peptide-amino acid

In an exemplary embodiment, at least one of R¹, R², R³, R⁴ and R⁵ possesses a phosphoramidite moiety. In another exemplary embodiment, the phosphoramidite moiety has a structure according to Formula IV:

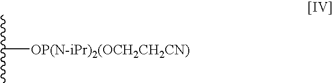

In a third aspect, the present invention is a nucleic acid having a sequence comprising at least one moiety having a structure of Formula I wherein the ring system A is a member selected from Formula V: 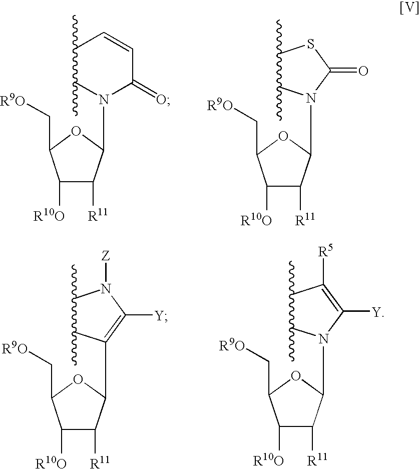
R⁹ and R¹⁰ are members independently selected from H, and a nucleic acid. The symbol R¹¹ is a member selected from H, OH, and a nucleic acid.

In an exemplary embodiment, the nucleic acid has a CpG format.

Synthesis of Analogs of TLR7 Ligands

Phagocytic white blood cells produce reactive oxidants that play critical roles in host defenses. These reactive oxidants, such as hypochlorous acid, nitrosyl chloride, and hydrogen peroxide, attack and oxidize nucleic acids. These oxidized nucleic acids have been observed at sites of inflammation and infection, and are thought to be signaling molecules for TLRs. Accordingly, in one aspect, the present invention provides TLR ligands and TLR ligand analogs based upon the structures formed by the attack of reactive oxidants on nucleic acids.

When the reactive oxidants attack nucleic acids containing purine bases, the damage generally involves alterations at the 8-position. Guanine is most susceptible to damage due to its intrinsically higher electron density at the available ring carbon atoms. Oxidized guanine derivatives have been observed at sites of inflammation and infection, including 8-oxo-, 8-bromo-, 8-chloro- and 8-nitroguanosine (Henderson et al., J. Biol. Chem., 276:7867-7875 (2001)). Additionally, 8-nitroguanosine acts as an endogenous TLR ligand, potentially through the activation of NF-kB. The present invention therefore provides 8-oxidized guanosines, which may play a role as endogenous ligands for TLR7.

8-oxidized guanosines are generally characterized by limited stability. For example, 8-nitrodeoxyguanosine in DNA is readily depurinated to produce 8-nitroguanine (Yermilov et al., FEBS Lett, 376:207-210, (1995)). In RNA, 8-nitroguanosine is more stable than the DNA analog, but is still considered a labile compound. In order to avoid depurination, 8-substituted non-purine ring systems could be prepared that were impervious to glycosidic cleavage. Thus, in another aspect, the invention provides compounds that include non-purine ring systems which are both stabile and isosteric with purines.

a) 7-Deazaguanosines (7DG)

The present invention provides 7-substituted, 8-substituted and 7,8-di substituted 7-deazaguanosines which can be produced through Scheme I:

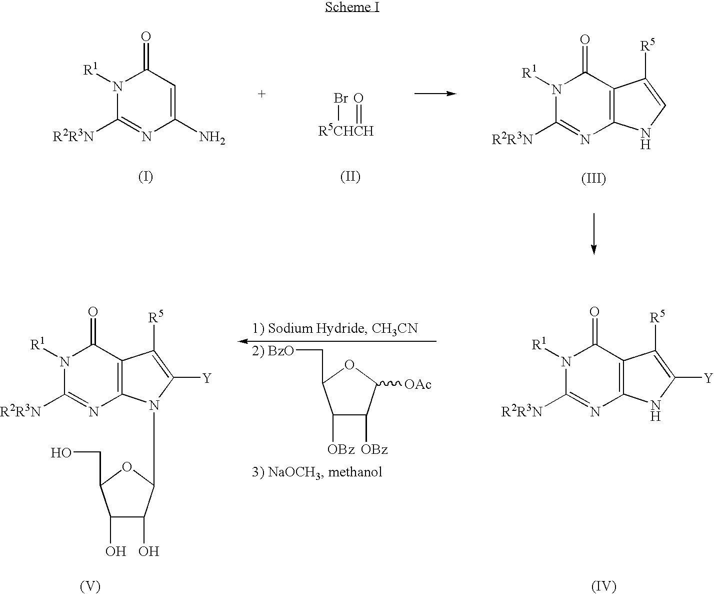

The 7-substituted-7-deazaguanine base is prepared by reacting 2-substituted amino-6-amino-4-pyrimidinone (I) with 3-substituted-2-bromo propanal (II) (Secrist et al., J. Org. Chem. 43:3937-3941 (1978)). Electrophilic aromatic substitution is then performed at the 8-position. The 7,8-disubstituted-7-deazaguanine (IV) is mixed with sodium hydride in acetonitrile, and then added to 1-O-acetyl-2,3,5-tri-O-benzoyl-D-ribofuranose in nitromethane in the presence of SnCl₄ to provide the benzyl-protected 7,8-disubstituted-7-deazaguanosine (Kazimierczuk et al., J. Am. Chem. Soc. 106:6379-6382 (1984)). Finally, sodium methoxide in methanol is added to deprotect the benzyl groups and provide the final product (V).

b) 9-Deazaguanosines (9DG)

Earlier studies (Girgis et al., J. Med. Chem., 33:2750-2755 (1990)) showed that C-nucleoside 9-deazaguanosine was not active in in vivo mouse models, which suggests little or no interferon induction by this derivative. However, addition of a halogen (chloro or bromo) atom at the 8-position resulted in compounds that were very active, suggesting that when there is a 7-nitrogen present, the 8-carbon is preferably oxidized (for example, with a halogen or oxygen) for acceptable immunoactivity.

The present invention provides 7-substituted, 8-substituted and 7,8-disubstituted 9-deazaguanosines according to Scheme II:

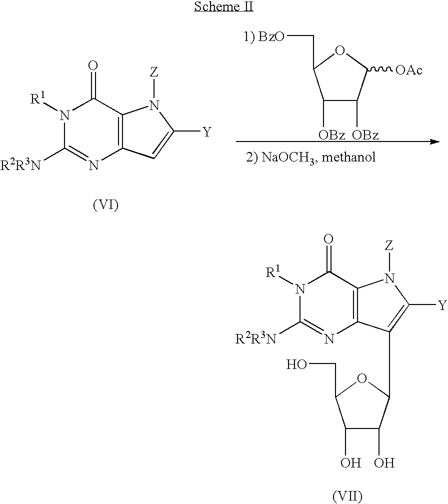

7,8-disubstituted-9-deazaguanine (VI) can be prepared, for example, by the methods of Klein (Klein et al., J. Org. Chem. 43:2536 (1978)) and Imai (Imai et al., Chem. Pharm. Bull. 12:1030 (1964)). 7,8-disubstituted-9-deazaguanine is then mixed with 1-O-acetyl-2,3,5-tri-O-benzoyl-D-ribofuranose in nitromethane in the presence of SnCl₄ to provide 2-amino-9-(2,3,5-tri-O-benzoyl-β-D-ribofuranosyl)-5H-pyrrolo[3,2-d]pyrimidin-4(3H)-one. Deprotection of the benzyl groups by treatment with sodium methoxide in methanol provides the final product (VII).

c) 7-thia-8-oxoguanosines and Their Analogs

7-thia-8-oxoguanosine (TOG) is one of the most potent innate immune system activators tested to date. It contains a sulfur in the 7-position, a relatively large atom which is known to occupy the space equivalent to two carbon atoms.

TOG can be produced using synthetic Scheme III:

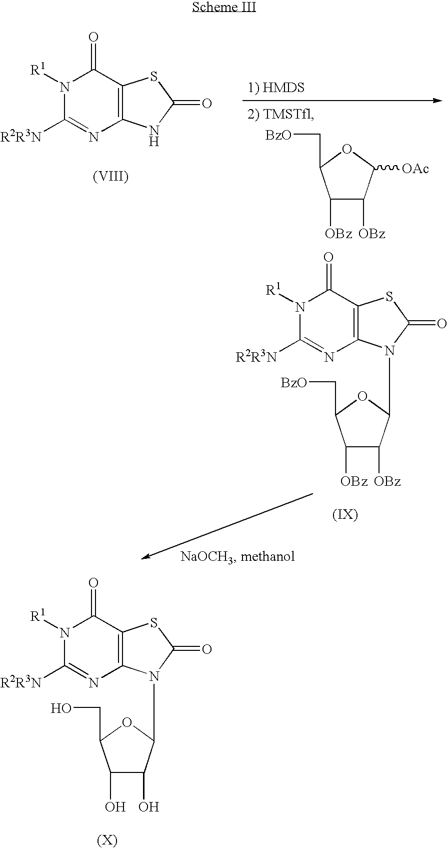

First, 5-aminothiazolo[4,5-d]pyrimidine-2,7(3H,6H)-dione (VIII) is prepared, for example, from commercially available 2,4 diamino-6-hydroxypyrimidine by the methods of Baker and Chatfield (Baker et al., J. Chem. Soc. C, 2478 (1970)). The product is then glycosylated by initial trimethylsilylation using hexamethyldisilazane followed by treatment with 1-O-acetyl-2,3,5-tri-O-benzoyl-D-ribofuranose in the presence of trimethylsilyl trifluoromethanesulfonate as a catalyst. Treating the major product, 5-amino-3-(2,3,5-tri-O-benzoyl-β-D-ribofuranosyl)thiazolo[4,5-d]pyrimidine-2,7(3H,6H)-dione (IX) with sodium methoxide in methanol provides the deprotected guanosine analogue, 5-amino-3-β-D-ribofuranosylthiazolo[4,5-d]pyrimidine-2,7(3H,6H)-dione (X).

As mentioned above, 8-oxidized guanosines, such as TOG, have limited stability. Replacing the 7-sulfur with two carbons yields the pyrido[2,3-d]pyrimidine ring system, a more stable TOG analog.

Pyrido[2,3-d]pyrimidine nucleosides can be produced through Scheme IV:

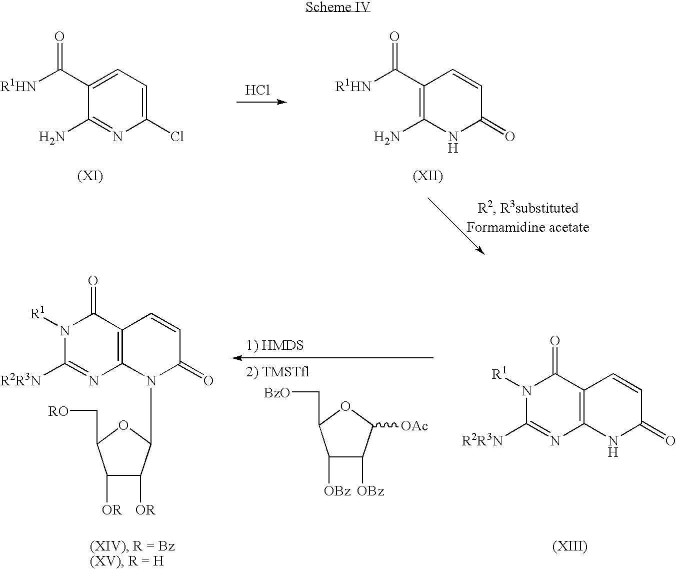

The “guanine-like” base can be prepared by treatment of the known 6-chloro-2-aminopyridine 3-carboxamide (XI) (Lamm, German Patent No. 2,605,467 (1977)) with aqueous HCl to provide the 2-pyridone derivative (XII). Ring closure of (XII) by fusion with formamidine acetate yields the guanine-like base (XIII). The nucleoside can then be prepared by performing the glycosylation using the commercially available ester-protected ribose in a manner similar to that reported for the preparation of TOG (Nagahara et al., J. Med. Chem. 33:407-415 (1990)). Finally, deprotection of the protected nucleoside (XIV) by ester cleavage using sodium methoxide in methanol yields the guanosine analog (XV) in the pyrido[2,3-d]pyrimidine ring system.

d) Non-purine Ring Systems with Alkyl and Alkenyl Groups

A large variety of alkyl and alkenyl derivatives of the non-purine ring systems can be prepared through direct alkylation of the sodium salts of the non-purine ring systems (See Scheme V below). Examples of non-purine ring systems include, but are not limited to, pyrido[2,3-d]pyrimidines, thiazolo[4,5-d]pyrimidines, and 7-deazapurines. This general method (see Scheme V) is also useful for the preparation of groups such as hydroxy- and alkoxy-substituted chains, whether straight or branched, as well as carbohydrates.

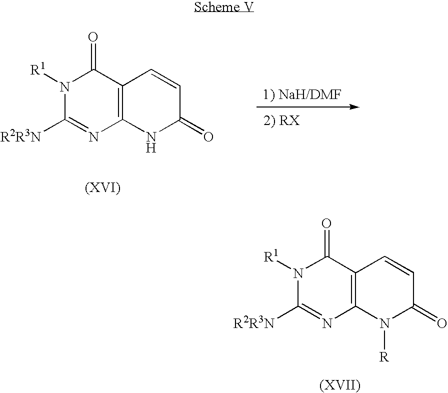

The general procedure follows that of Lewis, (Lewis et al., J. Heterocyc. Chem., 32:547-556 (1996)) wherein the guanine-like base (XVI) is treated with sodium hydride in anhydrous dimethyl formamide to form the sodium salt of (XVI). The appropriate alkyl halide (RX) can then be added to this solution and the mixture heated for 3-8 hours. Purification of the reaction mixture yields the corresponding alkylated product (XVII).

In another embodiment, the compounds include the alkyl moiety found in the immune stimulant R848 shown below, the dimethylethanol (or 2-hydroxy-2-methylpropyl) group. This group can be incorporated into (XVIII) by the same methodology as that used for (XVII) above, where RX is an allylic halide. Following attachment to the base, the resulting alkene can be hydrated under conditions of acid catalysis to afford the dimethylethanol group of (XIX) as shown in Scheme VI:

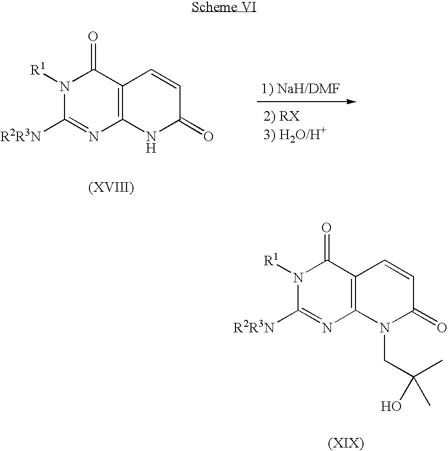

e) Nucleic Acids Incorporating TLR7 Ligands

The preparation of oligomers of the compounds of the invention can be accomplished by the following phosphoramidite procedure (Scheme VII) (Beaucage, et al., Tetrahedron, 48:2223-2311, (1992)).

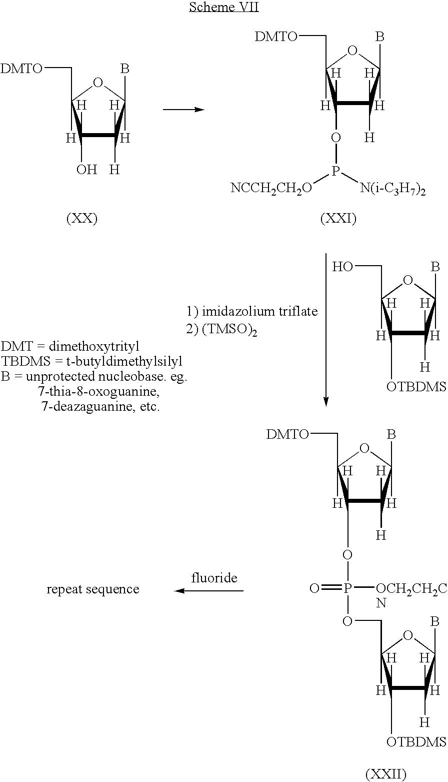

In the preparation of oligomers, a suitable nucleoside derivative (XX) can be phosphitylated, coupled to another derivatized nucleoside or nucleotide (XXI) and then oxidized with iodine or bis(trimethylsilyl)peroxide to the protected phosphate linkage (XXII), before repeating the process. Suitable derivatives for this procedure are those that can be selectively protected to allow for phosphitylation and coupling reactions which occur only on the desired OH function.

This general method may be used not only to prepare homopolymers but also heteropolymers by varying the type of nucleobase. For example, immune stimulatory sequence (ISS)-containing molecules may be prepared wherein one or more guanines are replaced with guanine analogs such as TOG, 7-deazaguanine, R848 and analogs thereof, while retaining the CpG motif. The rationale here is that since CpG molecules signal through Toll-like receptor 9 (TLR9) and the guanosine analogs signal through TLR7, a combination of the two types incorporated into one molecule could enhance immune stimulation to a greater degree than either type alone.

f) Alternative Cyclic Backbones.

The internucleotide linkage is normally a phosphate in the oligomers described above. However, a neutral backbone (having no net formal charge at physiological pH) may also be prepared which may affect the oligomer's stability and ability to penetrate into endosomes. One such example of a neutral backbone is based on the neutral glycerol unit that has been used in the preparation of non-nucleosidic coumarin derivatives (Wood et al., U.S. Pat. No. 6,005,093, Dec. 21, 1999) using phosphoramidite chemistry.

Nucleosides with acyclic backbones (with tris(trimethylsilyl)7-deazaguanosine shown as an example) can be produced through Scheme VIII:

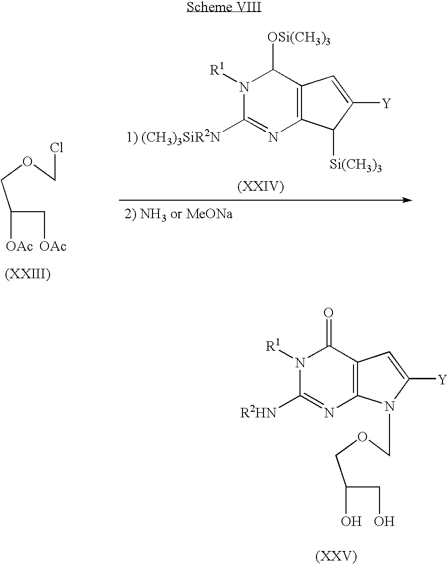

The starting material, 3-chloromethyl-1,2-di-O-acetyl-glycerol (XXIII), can be prepared from 1,2-di-O-acetyl-glycerol by a reported literature procedure (Gdendeev, J. Gen. Chem. (USSR), 6:1841 (1936)). (XXIII) can then be alkylated with tris(trimethylsilyl)-8-substituted-7-deazaguanosine (XXIV) in the presence of triethylamine in toluene. Removal of the silyl protecting groups by treatment with NH3 CH3—OH solution at room temperature overnight yields the final product (XXV).

Nucleoside and Nucleotide TLR Ligands

For several years, the immunostimulatory properties of bacterial DNA have been studied. CpG enriched oligonucleotides (ISS-ODN) that activate TLR9 have also been examined (Raz E (ed), Microbial DNA and Host Immunity, Humana Press, Totowa, N.J. (2002)). When administered simultaneously or up to two weeks before antigen, ISS-ODNs potently stimulate antigen-specific immune responses, which include T cell derived cytokines, antibodies and cytotoxic T cells. ISS-ODN can partially protect mice from infections with respiratory syncytial virus (RSV), Mycobacterium avium, and Listeria monocytogenes (Cho et al., J Allergy Clin Immunol, 108:697-702 (2001); Hayashi et al., Infect Immun, 69:6156-6164 (2001); Krieg et al., J Immunol, 161:2428-2434 (1998)). The protective effects of ISS-ODN may be orchestrated by TLR9-positive DC precursors that have been induced to differentiate into mature sentinel cells, poised to respond to infectious agents. Even a low frequency of DCs at mucosal sites, which have been instructed to differentiate by TLR9 activating ISS-ODN within endosomes, may be sufficient to increase host resistance for a period of several weeks.

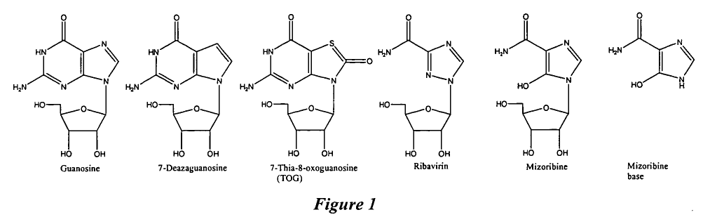

A series of guanosine congeners (including 7-thia-8-oxoguanosine [abbreviated TOG], 7-deazaguanosine, and 9-hexylguanine) (FIG. 1), which protected mice from lethal infection by interferon-sensitive viruses (e.g., Punta toro) (Smee et al., Can J Infect Dis 3(suppl B): 41B-48B (1992)), have been synthesized and analyzed. Compared to many other immune stimulating drugs, TOG and 7-deazaguanosine (7DG) are remarkably non-toxic to mice after systemic administration. It is also noteworthy that various 7-substituted 7-deazaguanosines (e.g., queuosine, epoxyqueuosine, and archaeosine) have been identified in tRNA (Frey et al., J Bacteriol, 170:2078-2082 (1988)). However, the guanosine congeners are insufficiently potent in people to justify clinical development as single agents. As noted earlier, TLR7 and TLR9 are co-expressed in the endosomes of DCs and B lymphocytes. Because phosphorothioate ISS-ODNs are taken up partly by endocytosis (Stein et al., Ciba Found Symp, 209:79-89 (1997)), and are resistant to nuclease degradation, these molecules can potentially achieve sustained contact with TLR9 within the endosome. In contrast, the cell permeable guanine nucleosides will not localize preferentially to endosomal vesicles, and will have a short half-life. Moreover, the specific activity of ISS-ODN rises by an order of magnitude when the ODNs aggregate. The multivalent ligands presumably form more stable complexes with the TLR9 receptor, thus maximizing signal transduction. In contrast, the guanine nucleoside and other ligands for TLR7 are almost certainly monovalent. Consequently, ODN-like molecules that incorporate more than one TLR7 activating nucleotide, either alone or in combination with a TLR9 activating CpG motif, should be synthesized and tested. Such a hybrid molecule could potentially bind to both TLR7 to TLR9, and hence more potently activate DCs. At the same time, a co-administered TLR8-L would potentiate the response of adjacent monocytes and macrophages to the products of DC activation.

Prodrugs of TLR Ligands

Research has also focused on the development of TLR ligands which are in the form of prodrugs. Prodrugs are composed of a prodrug portion covalently linked to an active therapeutic agent, such as a TLR-ligand or TLR-ligand analog. Prodrugs are capable of being converted to drugs (active therapeutic agents) in vivo by certain chemical or enzymatic modifications of their structure. Examples of prodrug portions are well-known in the art and can be found in the following references: Biological Approaches to the Controlled Delivery of Drugs, R. L. Juliano, New York Academy of Sciences, (1988); Hydrolysis in Drug and Prodrug Metabolism: Chemistry, Biochemistry, and Enzymology, Bernard Testa, Vch Verlagsgesellschaft Mbh, (2003); and Prodrugs: Topical and Ocular Drug Delivery, Kenneth Sloan, Marcel Dekker; (1992).

Thus, in an embodiment of the invention, the prodrug portion is covalently linked to the compounds of the invention. In another embodiment of the invention, the prodrug portion is covalently attached to a TLR-ligand or TLR-ligand analog. In still another embodiment of the invention, the prodrug portion is covalently attached to the heteroaryl portion of a TLR-ligand or TLR-ligand analog. In still another embodiment, the prodrug portion is covalently attached to an endocyclic amine of a heteroaryl portion of a TLR-ligand or TLR-ligand analog. In yet another embodiment, the R¹ substituent of the compound according to Formula I is a prodrug portion. In yet another embodiment, the Z substituent of the compound according to Formula I is a prodrug portion. In yet another embodiment, the R⁴ substituent of the compound according to Formula I is a prodrug portion. In still another embodiment, the prodrug portion is covalently attached to an exocyclic amine of a heteroaryl portion of a TLR-ligand or TLR-ligand analog. In yet another embodiment, the R² substituent of the compound according to Formula I is a prodrug portion. In yet another embodiment, the R³ substituent of the compound according to Formula I is a prodrug portion. In yet another embodiment, the prodrug portion is covalently attached to the sugar or sugar-analog portion of a TLR-ligand or TLR-ligand analog. In yet another embodiment, the prodrug portion is covalently attached to the ribose ring of a TLR-ligand or TLR-ligand analog. In yet another embodiment, the R⁹ substituent of the compound according to Formula V is a prodrug portion. In yet another embodiment, the R¹⁰ substituent of the compound according to Formula V is a prodrug portion. In yet another embodiment, the R¹¹ substituent of the compound according to Formula V is a prodrug portion.

The present invention provides prodrug versions of TLR-ligands and TLR-ligand analogs that impart stability to compounds, reduce their in vivo toxicity, or otherwise favorably affect their pharmacokinetics, bioavailability and/or pharmacodynamics. Examples of prodrug portions are peptides, e.g., peptides that direct the TLR ligand to the site of action, and a peptide which possesses two or more free and uncoupled carboxylic acids at its amino terminus. Other exemplary cleaveable prodrug portions include ester groups, ether groups, acyl groups, alkyl groups, phosphate groups, sulfonate groups, N-oxides, and tert-butoxy carbonyl groups.

It is generally preferred that in embodiments of the invention, the prodrug portion is cleaved, releasing the active therapeutic agent, once the drug is delivered to its site of action, or has cleared an important threshold to bioavailability, such as the blood/brain barrier or the digestive system. Thus, in one embodiment of the invention, the prodrug portions of the invention are traceless, such that once removed from the active therapeutic agent (such as during activation), no trace of the prodrug portion's presence remains. In another embodiment of the invention, the prodrug portions are characterized by their ability to be cleaved at a site in or near the target cell such as at the site of therapeutic action. Such cleavage is preferably enzymatic in nature. This feature aids in reducing systemic activation of the therapeutic agent, thereby reducing toxicity and systemic side effects.

The prodrug portions also serve to stabilize the therapeutic agent against degradation while in circulation. This feature provides a significant benefit since such stabilization results in prolonging the circulation half-life of the attached active therapeutic agent. The prodrug portion also serves to attenuate the activity of the attached active therapeutic agent so that the prodrug compound is relatively benign while in circulation and has the desired effect after activation at the desired site of action.

The stabilizing groups are preferably selected to limit clearance and metabolism of the active therapeutic agent by enzymes that may be present in blood or non-target tissue and are further selected to limit transport of the active therapeutic agent into the cells. The stabilizing groups serve to block degradation of the active therapeutic agent and may also act in providing other physical characteristics of the agent. The stabilizing group may also improve the active therapeutic agent's stability during storage in either a formulated or non-formulated form.

The present invention also relates to prodrug compounds that may be used for the treatment of disease, especially cancer and multiple sclerosis. Specifically, use of the prodrug portions described herein provide for prodrug compounds that display a high specificity of action, a reduced toxicity, and an improved stability in blood relative to compounds not containing such a prodrug structure.

The prodrug versions of TLR-ligands and TLR-ligand analogs of the present invention can be used for a variety of purposes. When the object of the experiment or therapy is to enhance interferon activation, a prodrug portion which is rapidly cleaved in vivo to give the active therapeutic agent is desired. Production of interferon can also be enhanced by administering this prodrug version of a TLR-ligand or TLR-ligand analog in combination with a high dosage of an IMPDH inhibitor over a short period of time.

When the object of the experiment or therapy is to inhibit cell proliferation, a prodrug portion which has greater stability in vivo and thus provides the active therapeutic agent over a longer period of time is desired. Cell proliferation can also be inhibited by administering this prodrug version of a TLR-ligand or TLR-ligand analog in combination with a low, continuous dosage of an IMPDH inhibitor.

Additional examples of prodrugs are described in a patent application entitled “New Uses for Inhibitors of Inosine Monophosphate Dehydrogenase”, Ser. No. 60/400,583, (Aug. 2, 2002). The entire disclosure of that application is incorporated herein by reference.

Administration of TLR Ligands

In another aspect, the present invention provides pharmaceutical compositions comprising a pharmaceutically acceptable excipient and a compound from the “Synthesis of analogs of TLR7 ligands” section provided above. In another aspect, the present invention provides pharmaceutical compositions comprising a pharmaceutically acceptable excipient and a compound of Formula I possessing a ring system according to Formula II. In another aspect, the present invention provides pharmaceutical compositions comprising a pharmaceutically acceptable excipient and a compound of Formula I possessing a ring system according to Formula V.

The compounds of the present invention can be prepared and administered in a wide variety of oral, parenteral and topical dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. Accordingly, the present invention also provides pharmaceutical compositions comprising a pharmaceutically acceptable carrier or excipient and either a compound from the “Synthesis of analogs of TLR7 ligands” section provided above, or a pharmaceutically acceptable salt of a compound from the “Synthesis of analogs of TLR7 ligands” section provided above.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents. In a preferred embodiment, a nucleic acid comprising the TLR ligands of the present invention is less than 30 nucleotides long.

Systemic administration of IMPDH inhibitors is described, for example, in U.S. Patent Application Nos. 60/400,583 and 60/400,568, both filed Aug. 2, 2002 and both of which are herein incorporated by reference.

Methods of Interacting with the Immune System by TLR Ligands

It is an object of the invention to illustrate methods in which a pharmaceutical composition, including a TLR ligand, are administered in a therapeutically effective amount in order to interact with the immune system. In one aspect, the pharmaceutical composition is employed in a method to activate the immune system of a mammal. In another aspect, the pharmaceutical composition is employed in a method to enhance resistance to infection in a mammal.

These aspects of the invention include, but are not limited to, the following embodiments. In an exemplary embodiment, the TLR ligand binds to a TLR expressed on an endosomal membrane.

In another exemplary embodiment, the pharmaceutical composition also comprises a CpG oligonucleotide (ISS-ODN). In another exemplary embodiment, the pharmaceutical composition also comprises an IMPDH inhibitor. In another exemplary embodiment, the pharmaceutical composition is administered to a mucus membrane.

In yet another exemplary embodiment, the TLR ligand is a homofunctional TLR ligand polymer. In another exemplary embodiment, the homofunctional TLR ligand polymer comprises a TLR ligand selected from the group consisting of a TLR-7 ligand and a TLR-8 ligand. In another exemplary embodiment, the homofunctional TLR ligand polymer comprises a TLR-7 ligand. In another exemplary embodiment, the TLR-7 ligand is a member selected from the group consisting of a 7-thia-8-oxoguanosinyl (TOG) moiety, a 7-deazaguanosinyl (7DG) moiety, and an imiquimod moiety.

In an exemplary embodiment, the homofunctional TLR ligand polymer comprises a TLR-8 ligand. In another exemplary embodiment, the TLR-8 ligand is a resiquimod moiety.

In another exemplary embodiment, the TLR ligand is a heterofunctional TLR ligand polymer. In another exemplary embodiment, the heterofunctional TLR ligand polymer comprises a TLR-7 ligand and a member selected from the group consisting of a TLR-8 ligand and a TLR-9 ligand. In another exemplary embodiment, the heterofunctional TLR ligand polymer comprises a TLR-7 ligand, a TLR-8 ligand, and a TLR-9 ligand. In another exemplary embodiment, the heterofunctional TLR ligand polymer comprises a TLR-8 ligand and a TLR-9 ligand.

In an exemplary embodiment, the infection is caused by a virus. In another exemplary embodiment, the virus is an interferon-sensitive virus. In another exemplary embodiment, the infection is caused by a bacteria. In another exemplary embodiment, the bacteria causes an intracellular bacterial infection. In another exemplary embodiment, an antibiotic is also administered to the mammal.

Methods of Using IMPDH Inhibitors in Combination with TLR Ligands

In an exemplary embodiment, inosine monophosphate dehydrogenase (IMPDH) inhibitors are used in combination with TLR ligands in order to activate the immune system or to enhance resistance to infection, or to treat diseases such as viral or bacterial infections, cancer, and autoimmune diseases. In another exemplary embodiment, IMPDH inhibitors enhance interferon production induced by TLR ligands (e.g., TLR 7 or 8 ligands).

Currently, three IMPDH inhibitors are used clinically: ribavirin, mizoribine, and mycophenolate mofetil. Ribavirin and mizoribine are prodrugs that are phosphorylated intracellularly to produce IMP analogs (Goldstein et al., Cuff Med Chem, 6:519-536 (1999)). Viramidine is a prodrug of Ribavirin. Mycophenolate mofetil is immunosuppressive, and has gastrointestinal irritative properties that may be attributable to its enterohepatic recirculation (Papageorgiou C, Mini Rev Med Chem., 1:71-77 (2001)). Mizoribine aglycone, a prodrug, is used as an IMPDH inhibitor. Other IMPDH inhibitors are Other IMPDH inhibitors, including prodrugs of mizoribine and mizoribine aglycone are known and are described in U.S. Patent Application Nos. 60/400,583 and 60/400,568, both of which are herein incorporated by reference.

Other IMPDH inhibitors include Tiazofurin. Tiazofurin is anabolized to become an NAD analog that inhibits IMPDH. Tiazofurin may be prepared as described in U.S. Pat. No. 4,680,285 or U.S. Pat. No. 4,451,648, incorporated herein by reference. Selenazofurin, benzamide riboside, 6-CL-IMP, and VX-497 are also IMPDH inhibitors.

In a preferred embodiment, IMPDH inhibitors are used to enhance induction of interferon synthesis by TLR ligands. That is, while both IMPDH inhibitors and TLR ligands are able to activate interferon regulatory factors, only TLR ligands appear to activate or induce interferon sythesis on their own. However, in the presence of IMPDH inhibitors, induction of interferon synthesis by TLR ligands is enhanced. Both synthetic (e.g., guanosine cogeners) and naturally occurring TLR ligands (e.g., virus and viral products) show enhancement of interferon synthesis in the presence of IMPDH inhibitors

Both ribavirin and mizoribine have in vitro antiviral activity (Hosoya et al., J Infect Dis, 168:641-646 (1993)). Ribavirin is approved for the treatment of respiratory syncytial virus (RSV) pneumonitis, and chronic hepatitis (together with interferon-α). The biological bases for the antiviral actions of ribavirin are controversial, and include mutagenic incorporation into viral nucleic acid, and interference with mRNA cap formation. However, several literature reports indicate that ribavirin enhances the synthesis of interferon and Th1-type cytokines in response to viral infection, while actually blocking the synthesis of TNF-α (Hultgren et al., J Gen Virol, 79:2381-2391 (1998); Ning et al., J Immunol, 160:3487-3493 (1998)). Because most microbial pathogens contain one or more PAMPs, the nucleoside-like IMPDH inhibitors can thus act to enhance signal transduction in response to TLR activation, while reducing side effects attributable to TNF-α release.

Ribavirin, mizoribine, and mizoribine base potentiate cytokine and co-stimulatory molecule production by mononuclear leukocytes exposed to synthetic TLR ligands. These effects are cell-specific (they do not occur in lymphocytes), and are attributable, at least in part, to activation of downstream interferon regulatory factors (IRFs), particularly IRF-1, IRF-3 and IRF-7. The latter two IRFs have been demonstrated to play distinct and essential roles in interferon α and β gene induction by viruses (Sato et al., Immunity, 13:539-548 (2000)). The IRF-stimulating actions of the drugs are attributable to IMPDH inhibition, since they are not seen in guanine-supplemented medium. It seems likely that GTP depletion in macrophages and/or dendritic cells induces a canonical “stress response” that leads to IRF-1, IRF-3 and 7 activation.

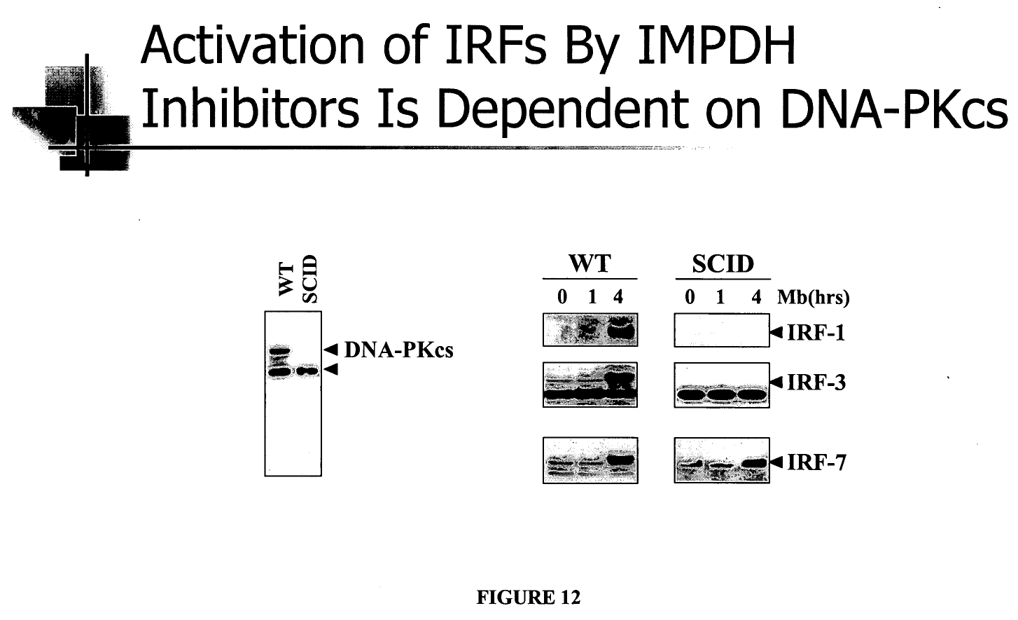

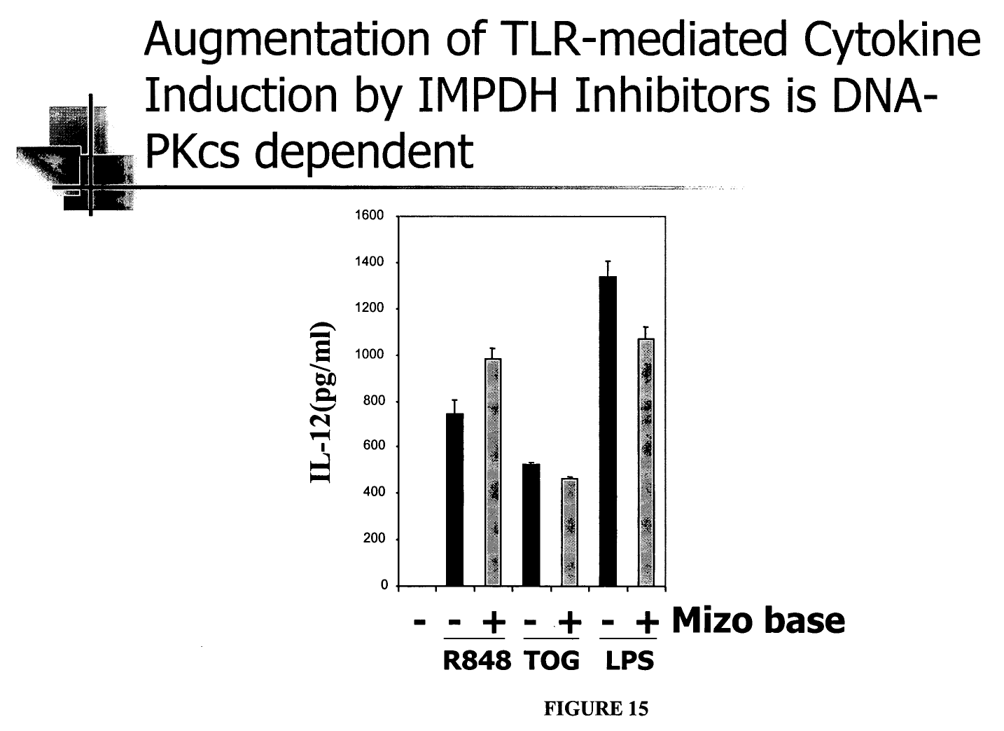

Activation of IRFs by IMPDH inhibitors and TLR7 ligands is dependent on DNA dependent protein kinase (DNA-PK). (See, e.g., FIGS. 12-14.) In addition the enhancement of TLR7 mediated induction of cytorkine production, including intererfon production, is also DNA-PK dependent. (See, e.g., FIG. 15.)

Methods of Detecting Activation of Innate Immunity

Those of skill will recognize that there are a variety of ways to detect activation of innate immunity. Innate immunity is activated as a result of activation of TLR molecules. Thus, one method to detect activation of innate immunity is detection of TLR signaling. For example, the transcription factor NFkB is activated to induce transcription as a result of TRL signaling.

Innate immunity also results in expression of cytokines and co-stimulatory molecules. Exemplary cytokines include TNFα, IL-12, IFNα, IFNβ and IFNγ. Exemplary co-stimulatory molecules include CD40, CD80, and CD86 expressed on, for example, F4/80+ splenocytes, CD14+ macrophages, or CD11+ splenocytes.

Methods of Detecting Activation of Adaptive Immunity

Those of skill will recognize that there are a variety of ways to detect activation of adaptive immunity.

Specific activation of CD4+ or CD8+ T cells may be detected in a variety of ways. Methods for detecting specific T cell activation include, but are not limited to, detecting the proliferation of T cells, the production of cytokines (e.g., lymphokines), or the generation of cytolytic activity (i.e., generation of cytotoxic T cells specific for a Her-2/neu fusion protein). For CD4+ T cells, a preferred method for detecting specific T cell activation is the detection of the proliferation of T cells. For CD8+ T cells, a preferred method for detecting specific T cell activation is the detection of the generation of cytolytic activity.

Detection of the proliferation of T cells may be accomplished by a variety of known techniques. For example, T cell proliferation can be detected by measuring the rate of DNA synthesis. T cells which have been stimulated to proliferate exhibit an increased rate of DNA synthesis. A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca2+ flux, or dye uptake, such as 3-(4,5-dimethylthiazol-2-yl)-2,5-diphenyltetrazolium. Alternatively, synthesis of lymphokines (e.g., interferon-gamma) can be measured or the relative number of T cells that can respond to intact antigen protein may be quantified.

Adaptive immunity is also characterized by proliferation of B cells that produce antibodies directed against a specific antigen.

Methods of Enhancing Resistance to Viral and Bacterial Infection by Administration of TLR-Ligands.

Synthetic TLR ligands can be used to protect an individual from viral or bacterial infection by activating the immune system. In some embodiments, the innate immune system is activated by administration of synthetic TLR ligands. In other embodiments, the adaptive immune system is activated by TLR ligands. TLR lignads can be administered in combination with IMPDH inhibitors to enhance resistance to viral or bacterial infection.

Innate immune system activation plays an important role in host defenses against bacterial as well as viral pathogens. An ideal agent(s) for the protection of the public must stimulate both an antiviral and an antibacterial state. However, most in vivo experiments with synthetic immunostimulatory molecules have been directed toward interferon-sensitive viruses.

It is demonstrated in Example 4 that the TLR9 ligand ISS-ODN can diminish the replication of Mycobacterium avium in macrophages, and can protect mice from lethal infection with Listeria monocytogenes. Example 4 additionally shows that ISS-ODN significantly potentiates the ability of antibiotics to eliminate intracellular bacteria in macrophages. The TLR ligands can act to enhance a host's bacteriocidal activity, while the antibiotics can act to slow bacterial replication. The observed synergistic effects of the TLR ligands with antibiotics are potentially attributable to induction of both indoleamine dioxygenase (leading to tryptophan depletion), and nitric oxide synthase.

In some embodiments, infection of intracellular bacteria are treated with TLR ligands, alone or in combination with IMPDH inhibitors. Intracellular bacteria include both facultative and obligate intracellular bacteria. Exemplary intracellular bacteria include Mycobacterium tuberculosis, Mycobacterium bovis, Mycobacterium leprae, Salmonella enterica, Brucella, Legionella pneumopholia, Listeria moncyotgenes, Francisella tularensis, Rickettsia rickettsii, Rickettsia prowazeki, Rickettsia typhi, Rickettsiatsutsujamushi, Coxiella burnetii, Chlamydia trachomatis, Chlamydia psittaci, Chlamydia pneumoniae, Shigella, Yersinia, and Toxoplasma gondii.

In contrast to the immediate and non-specific nature of innate immunity, adaptive immunity is antigen-specific and results in immune memory. Some combinations of synthetic TLR ligands will enhance maturation of DCs, which consequently will orchestrate CD4 and CD8 responses. A highly sensitive and reproducible in vitro screening system will provide a rapid means of assessing the ability of TLR-L, their combinations, with or without IMPDH inhibitors to enhance CD4+ and CD8+T cell activation. The most potent combinations are useful as adjuvants in vivo.

Treatment of Viral Infections Using TLR Ligands and IMPDH Inhibitors, Separately or in Combination.

TLR ligands of the present invention are useful for treating viral infections. In some embodiments an IMPDH inhibitor is administered with the TLR ligand. The administration of the IMPDH inhibitor will depend on the needs of the user and the type of viral infection.

RNA virus can act as natural TLR ligands, and activate a TLR (e.g., TLR-3). IMPDH inhibitors can be used to treat such RNA virus infections, thereby enhancing cellular production of interferon in response to the viral infection. In addition to the natural TLR ligands provided by the RNA virus, synthetic TLR ligands of the present invention can also be administered in combination with IMPDH inhibitor to treat the RNA virus infection. RNA virus includes virus of the following families: Picornaviridae, Flaviviridae, Caliceviridae, Astroviridae, Togaviridae, Nodaviridae, Tetraviridae, Coronaviridae, Ateriviridae, Birniviridae, Reoviridae, Rhabdoviridae, Filoviridae, Bornaviridae, Paramyxoviridae, Bunyaviridae, Orthamyxoviridae, Delta virus, and Arenaviridae. Preferred RNA virus include Hepatitis C Virus (HCV) and the Coronavirus that causes Severe Acute Respiratory Syndrome (SARS). The IMPDH inhibitor can be adminstered directly to the site of infection in some embodiments. For example, for treatment of the Coronavirus that causes SARS or other virus that infect the respiratory tract, IMPDH inhibitors can be administered by inhalation therapy directly to the lung, preferably in a high concentration. From 1-100 mg/ml IMPDH inhibitor in a physiological saline solution could be adminstered in this manner.

In another preferred embodiment, the RNA virus has mutated with the result that cellular production of interferon is diminished. This type of virus can be treated with IMPDH inhibitors alone or in combination with the synthetic TLR ligands of the present invention. Examples of RNA virus that have mutated and cause diminished interferon induction include HCV and the Coronavirus that causes SARS.

The TLR ligands of the present invention can be used alone or in combination with IMPDH inhibitors to treat infections caused by DNA virus. In a preferred embodiment the IMPDH inhibitor is administered systemically. DNA virus includes Papovaviridae, Adenoviridae, Hepadenoviridae, Herpesviridae, Poxviridae, and Parvoviridae. Hepatitis B virus is a preferred DNA virus for the treatment methods of the present invention.

The invention also provides methods for treatment of viral diseases using a combination of Type I interferon and a member selected from mizoribine, mizoribine base, mizoribine aglycone, an enantiomer of such a compound, a prodrug of such a compound, a pharmaceutically acceptable salt of such a compound, and combinations thereof, given in therapeutically effective doses. Preferred virus for such treatment include a coronavirus that causes Severe Acute Respiratory Syndrome (SARS), a Hepatitis B virus, and a Hepatitis C Virus.

Methods for the Treatment of Disease by Administration of TLR-ligands

It is an object of the invention to illustrate methods in which a pharmaceutical composition, including an interferon inducer and a member selected from an inhibitor of inosine monophosphate dehydrogenase (IMPDH), an enantiomer of such a compound, a prodrug of such a compound, a pharmaceutically acceptable salt of such a compound, and combinations of these compounds, is administered in a therapeutically effective amount in order to treat disease. In one aspect, the disease in cancer. In another aspect, the disease is an autoimmune disease. In another embodiment, an interferon inducer, an IMPDH inhibitor and a Type I interferon are administered to treat disease (i.e., cancer or an autoimmune disease).

These aspects of the invention include, but are not limited to, the following embodiments. In an exemplary embodiment, the interferon inducer includes a therapeutically effective amount of a pharmaceutical composition which can contain a nucleic acid which can be a TLR ligand. In another exemplary embodiment, the TLR ligand binds to a TLR expressed on an endosomal membrane.

In another exemplary embodiment, the pharmaceutical composition also comprises a CpG oligonucleotide (ISS-ODN). In another exemplary embodiment, the pharmaceutical composition is administered to a mucus membrane.

In yet another exemplary embodiment, the TLR ligand is a homofunctional TLR ligand polymer. In another exemplary embodiment, the homofunctional TLR ligand polymer comprises a TLR ligand selected from the group consisting of a TLR-7 ligand and a TLR-8 ligand. In another exemplary embodiment, the homofunctional TLR ligand polymer comprises a TLR-7 ligand. In another exemplary embodiment, the TLR-7 ligand is a member selected from the group consisting of a 7-thia-8-oxoguanosinyl (TOG) moiety, a 7-deazaguanosinyl (7DG) moiety, a resiquimod moiety (R848), and an imiquimod moiety.

In an exemplary embodiment, the homofunctional TLR ligand polymer comprises a TLR-8 ligand. In another exemplary embodiment, the TLR-8 ligand is a resiquimod moiety.

In another exemplary embodiment, the TLR ligand is a heterofunctional TLR ligand polymer. In another exemplary embodiment, the heterofunctional TLR ligand polymer comprises a TLR-7 ligand and a member selected from the group consisting of a TLR-8 ligand and a TLR-9 ligand. In another exemplary embodiment, the heterofunctional TLR ligand polymer comprises a TLR-7 ligand, a TLR-8 ligand, and a TLR-9 ligand. In another exemplary embodiment, the heterofunctional TLR ligand polymer comprises a TLR-8 ligand and a TLR-9 ligand.

Methods for the Treatment of Cancer by Administration of TLR-ligands

In an exemplary embodiment for treating cancer, the cancer is an interferon-sensitive cancer. In another exemplary embodiment for treating cancer, the interferon-sensitive cancer can be a leukemia, a lymphoma, a myeloma, a melanoma, or a renal cancer.

The present invention provides a method of treating cancer by administering polymers containing TLR ligands or other compounds that induce interferon (INF) production. As indicated above, TLR ligands activate the innate immune system. Additionally, TLR ligands can induce interferon production. IMPDH inhibitors enhance the induction of interferon production by TLR ligands and are thus given in combination with an interferon inducer to treat cancer.

Interferons are a family of proteins. Four classes of interferon molecules are currently known: INF-α, INF-β, INF-γ, and INF-ω. INF-α and INF-β are also known as Type I interferons. Interferon proteins are now in use as therapeutic agents for cancer treatment. Antitumor effects of INF proteins can result from a direct effect on tumor cells, such as inhibition or decrease in rate of proliferation or induction of apoptosis; an indirect effect on immune effector populations that interact with tumor cells; or from inhibition of angiogenesis. Cancers that are amenable to treatment with INF's include leukemia, melanoma, renal cell cancer, myeloma, lymphoma, follicular cancer, T-cell cancer, multiple myeloma, midgut carcinoids, Kaposi's sarcoma, ovarian, basal cell, bladder, and breast cancer.

In addition, inducers of INF expression can also be used to treat cancer. Activation of the innate immune system, for example by TLR-ligands, induces cellular INF production. Synthetic inducers of INFs are known and include double-stranded polynucleotides, tilorone, halopyrimidines, acridines, substituted quinolones, and flavone acetic acid.

Induction of interferon expression can be determined using a variety of methods. One methodology examines actual production of INF. For instance, amounts of INF mRNA and INF proteins in the cell can be monitored. Because INF proteins are secreted, INF protein levels can also be assayed outside of the cell. A second methodology does not look for INF production, but rather determines INF induction by assaying INF's function on other entities. For example, INF's can induce expression of hundreds of genes, including PKR, PML, RAP46/Bag-1, phospholipidscramblase, 2-5A synthetase, indoleamine 2,3-dioxygenase, and IFN regulatory factors. Levels of the interferon induced gene products (e.g., mRNA and proteins) can be measured to determine induction of interferon. In addition, other downstream effects of interferon (e.g., inhibition or decrease in rate of proliferation or induction of apoptosis; effects on immune effector populations that interact with tumor cells; and from inhibition of angiogenesis) can be assayed to determine interferon induction.

The invention also provides methods for treatment of cancer using a combination of Type I interferon and a member selected from mizoribine, mizoribine base, mizoribine aglycone, an enantiomer of such a compound, a prodrug of such a compound, a pharmaceutically acceptable salt of such a compound, and combinations thereof; given in therapeutically effective doses. Preferred cancer for such treatment include a leukemia, a lymphoma, a myeloma, a melanoma, and a renal cancer.

Treatment of Autoimmune Disease by Administration of TLR Ligands

Synthetic TLR ligands can be used to treat autoimmune, alone or in combination with IMPDH inhibitors. In one embodiment, synthetic TLR ligands can be used to enhance the amount of interferon beta synthesized by a subject, thereby providing a therapy for autoimmune disease. In another embodiment, synthetic TLR ligands can be used to decrease the amount of interferon gamma synthesized by a subject. Synthetic TLR ligands can be used to treat autoimmune disease alone, or in combination with interferon beta. In addition, inducers of interferon expression can also be used to treat autoimmune disease in combination with synthetic TLR ligands. Activation of the innate immune system, for example by TLR-ligands, induces cellular interferon expression. Synthetic inducers of interferons are known and include double-stranded polynucleotides, tilorone, halopyrimidines, acridines, substituted quinolones, and flavone acetic acid. IMPDH inhibitors, as defined above, enhance induction of interferon expression by TLR ligands and thus, are used in combination with TLR ligands to treat autoimmune diseases.

Autoimmune disease, including multiple sclerosis are a group of illnesses generally understood to be caused by the over-production of cytokines, lymphotoxins and antibodies by white blood cells, including in particular T-cells. During an autoimmune reaction, T-cells are understood to release chemical mediators such as interferon gamma, which lead to the development of pathological symptoms of autoimmune reaction. A treatment for autoimmune disease may therefore involve the use of agents capable of inhibiting release of interferon gamma from T-cells. Agents that inhibit interferon gamma include type I interferons, i.e., INF-α and INF-β. Autoimmune diseases include diabetes, rheumatoid arthritis, multiple sclerosis, lupus erythematosis, myasthenia gravis, scleroderma, Crohn's disease, ulcerative colitis, Hashimoto's disease, Graves' disease, Sjogren's syndrome, polyendocrine failure, vitiligo, peripheral neuropathy, graft-versus-host disease, autoimmune polyglandular syndrome type I, acute glomerulonephritis, Addison's disease, adult-onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barre syndrome, hemochromatosis, Henoch-Schonlein purpura, idiopathic IgA nephropathy, insulin-dependent diabetes mellitus (IDDM), juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome and thyroiditis.

Multiple sclerosis is an example of an autoimmune disease that is amenable to treatment by increasing levels of a Type I interferon. Interferon beta is a cytokine that has therapeutic application in the treatment of a variety of autoimmune diseases. In autoimmune disease such a s MS, the activation of Th1 type T-cells is thought to be a primary component of the autoimmune response. In MS, the autoimmune response attacks the myelin sheath neuronal axons. One of the classical markers of Th1 cell activation is the production of interferon gamma. In the development of interferon beta as a therapeutic agent for the treatment of MS, studies were conducted to demonstrate the ability of interferon gamma to decrease the rate of production of interferon gamma from lymphocytes in vitro. (Ann. Neurol. 44:27-34 (1998) and Neurology 50:1294-1300 (1998)). The reduction of interferon gamma release by treatment with interferon beta is an indication of the effectiveness of interferon beta in the treatment of MS. There is a continuing need for other agents that inhibit the production of interferon gamma, particularly agents for use in the treatment of autoimmune disease, including agents that may work synergistically to enhance the effect of existing agents such as interferon beta.

Those of skill will recognize that treatment of MS using synthetic TLR ligands can result in reduction or elimination of the symptoms of MS, e.g., loss of vision, double vision, dizziness, weakness, loss of sensation, problems controlling bladder and bowel function, muscle weakness in their extremities and difficulty with coordination and balance, paresthesias, transitory abnormal sensory feeling such as numbness or “pins and needles,” pain, and cognitive impairments such as difficulties with concentration, attention, memory, and judgment.

Those of skill will be able to determine whether synthetic TLR ligands decrease production of interferon gamma. For example, T-cells can be isolated and stimulated to produce interferon gamma using concanavalin A. This system can be used to determine the effect of TLR ligands, alone or in the presence of interferon beta or interferon inducers, on interferon gamma production. See e.g., Clark-Lewis, WO 99/47158; herein incorporated by reference.

The invention also provides methods for treatment of autoimmune diseases using a combination of Type I interferon and a member selected from mizoribine, mizoribine base, mizoribine aglycone, an enantiomer of such a compound, a prodrug of such a compound, a pharmaceutically acceptable salt of such a compound, and combinations thereof; given in therapeutically effective doses. Preferred virus for such treatment include a coronavirus that causes Severe Acute Respiratory Syndrome (SARS), a Hepatitis B virus, and a Hepatitis C virus.

Treatment of Diseases with Topical Interferon Inducers and IMPDH Inhibitors.

The present invention provides methods to treat diseases accessible to topical treatment with topical interferon inducers and IMPDH inhibitors. The interferon inducer can be a TLR ligand and in preferred embodiments is chosen from the following group: resiqumod, imiquimod, ISS-ODN, and the nucleic acid polymers of the present invention. The IMPDH inhibitor can be given systemically or topically.

Disease that are accessible to topical treatment include some cancers and precancerous conditions. Preferred cancers include melanoma, superficial bladder cancer, actinic keratoses, intraepithelial neoplasia, and basal cell skin carcinoma. Preferred precancerous conditions include actinic keratoses and intraepithelial neoplasia. Some infections caused by virus are also accessible to topical treatment and thus can treated with topical interferon inducers and IMPDH inhibitors. Preferred viral infections for treatment include human papilloma virus infection, molluscum contagiosum, and herpes virus infection.

Treatment of Crohn's Disease with Probiotics and IMPDH Inhibitors.

The present invention also provides a method of treating Crohn's Disease by administering a member selected from an inhibitor of inosine monophosphate dehydrogenase (IMPDH), and a member selected from the group consisting of probiotics and glycolipids, i.e., a natural TLR ligand.

Activation of the Immune System by TLR Ligands

Those of skill will recognize methods to determine whether synthetic TLR ligands activate an immune response. For example, activation of the immune response can be determined by assaying changes in gene expression that typically follow immune system activation. Gene expression can be determined by measuring mRNA levels or protein levels of interest. In some embodiments levels of cytokines will be determined. Cytokine levels can increase or decrease with immune system activation. In a preferred embodiment levels of an interferon mRNA or protein are used to determine immune system activation. Those of skill will recognize that the same methods can be used to determine the ability of IMPDH inhibitors to enhance INF production by TLR ligands. Immune system activation can result in activation of the innate immune system, or the adaptive immune system, or both.

After administration of TLR ligands, a mammal can respond by altering expression of gene products that are directly or indirectly regulated by TLR signaling. Gene expression can increase or decrease. Gene products include, for example, mRNA and proteins. Levels of mRNA and proteins can be measured to determine if gene expression has changed. In the case of enzymes, enzymatic activity can be used to determine expression levels.

Amplification-based assays can be used to detect the presence of interferon nucleic acid in a sample. In such amplification-based assays, the nucleic acid sequences act as a template in an amplification reaction (e.g. Polymerase Chain Reaction (PCR). In a quantitative amplification, the amount of amplification product will be proportional to the amount of template in the original sample. Comparison to appropriate (e.g. healthy tissue) controls provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). The known nucleic acid sequence for the genes is sufficient to enable one of skill to routinely select primers to amplify any portion of the gene.

Real time PCR is another amplification technique that can be used to determine gene copy levels or levels of mRNA expression. (See, e.g., Gibson et al., Genome Research 6:995-1001, 1996; Heid et al., Genome Research 6:986-994, 1996). Real-time PCR is a technique that evaluates the level of PCR product accumulation during amplification. This technique permits quantitative evaluation of mRNA levels in multiple samples. For gene copy levels, total genomic DNA is isolated from a sample. For mRNA levels, mRNA is extracted from tumor and normal tissue and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, β-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Other suitable amplification methods include, but are not limited to ligase chain reaction (LCR) (see Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR.

Gene expression levels can also be assayed as a marker TLR ligand activity or including induction of regulated gene products. In preferred embodiments, activity of the interferon gene is determined by a measure of gene transcript (e.g. mRNA), by a measure of the quantity of translated protein, or by a measure of gene product activity.

Methods of detecting and/or quantifying the gene transcript (mRNA or cDNA) using nucleic acid hybridization techniques are known to those of skill in the art (see Sambrook et al. supra). For example, one method for evaluating the presence, absence, or quantity of mRNA involves a Northern blot transfer.

The probes can be full length or less than the full length of the nucleic acid sequence encoding the protein. Shorter probes are empirically tested for specificity. Preferably nucleic acid probes are 20 bases or longer in length. (See Sambrook et al. for methods of selecting nucleic acid probe sequences for use in nucleic acid hybridization.) Visualization of the hybridized portions allows the qualitative determination of the presence or absence of mRNA.

In another preferred embodiment, a transcript (e.g., mRNA) can be measured using amplification (e.g. PCR) based methods as described above. In a preferred embodiment, transcript level is assessed by using reverse transcription PCR (RT-PCR). In another preferred embodiment, transcript level is assessed by using real-time PCR.

The expression level of an interferon gene can also be detected and/or quantified by detecting or quantifying the expressed interferon polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like. Immunohistochemical methods can also be used to detect interferon protein. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu et al. (1980) Am. J. Clin. Path. 75:734-738. The isolated proteins can also be sequenced according to standard techniques to identify polymorphisms.

A polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled anti-antibody. Alternatively, the labeling agent may be a third moiety, such as another antibody, that specifically binds to the antibody/polypeptide complex.

In one preferred embodiment, the labeling agent is a second human antibody bearing a label. Alternatively, the second antibody may lack a label, but it may, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin. In some embodiments, Western blot analysis is used to detected and or quantify interferon protein.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Proteins can be detected and/or quantified in cells using immunocytochemical or immunohistochemical methods. IHC (immunohistochemistry) can be performed on paraffin-embedded tumor blocks using a interferon-specific antibody. IHC is the method of colorimetric or fluorescent detection of archival samples, usually paraffin-embedded, using an antibody that is placed directly on slides cut from the paraffin block. To detect and/or quantify interferon in, for example tissue culture cells or cells from a subject that are not embedded in paraffin (for example, hematopoetic cells) ICC (immunocytochemistry) can be used. ICC is like IHC but uses fresh, non-paraffin embedded cells plated onto slides and then fixed and stained.

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of peptides can be determined by conventional techniques to determine the magnitude of the antibody response of an animal that has been immunized with the peptide. Generally, the peptides that are used to raise the anti-peptide antibodies should generally be those which induce production of high titers of antibody with relatively high affinity for the polypeptide.

Preferably, the antibodies produced will be monoclonal antibodies (“mAb's”). For preparation of monoclonal antibodies, immunization of a mouse or rat is preferred. Polyclonal antibodies can also be used.

It is also possible to evaluate an mAb to determine whether it has the same specificity as a mAb of the invention without undue experimentation by determining whether the mAb being tested prevents a mAb of the invention from binding to the subject gene product isolated as described above. If the mAb being tested competes with the mAb of the invention, as shown by a decrease in binding by the mAb of the invention, then it is likely that the two monoclonal antibodies bind to the same or a closely related epitope. Still another way to determine whether a mAb has the specificity of a mAb of the invention is to preincubate the mAb of the invention with an antigen with which it is normally reactive, and determine if the mAb being tested is inhibited in its ability to bind the antigen. If the mAb being tested is inhibited then, in all likelihood, it has the same, or a closely related, epitopic specificity as the mAb of the invention.

Synthetic TLR ligands can be used to activate the innate immune system. Those of skill will recognize that there are a variety of ways to detect activation of innate immunity. Innate immunity is activated as a result of activation of TLR molecules. Thus, one method to detect activation of innate immunity is detection of TLR signaling. For example, the transcription factor NFkB is activated as a result of TRL signaling. TLR7 ligands and guanosine cogeners also activate DNA-dependent protein kinase, which in turn, appears to activate IRFs.

Innate immunity also results in expression of cytokines and co-stimulatory molecules. Exemplary cytokines include TNFα, IL-12, IFNα, and IFNγ. Exemplary co-stimulatory molecules include CD40, CD80, and CD86 expressed on, for example, F4/80+ splenocytes, CD14+ macrophages, or CD11+ splenocytes.

Synthetic TLR ligands can be used to activate the adaptive immune system. Those of skill will recognize that there are a variety of ways to detect activation of adaptive immunity. In some embodiments synthetic TLR ligands are used as adjuvants to activate adaptive immununity.

Specific activation of CD4+ or CD8+ T cells may be detected in a variety of ways. Methods for detecting specific T cell activation include, but are not limited to, detecting the proliferation of T cells, the production of cytokines (e.g., lymphokines), or the generation of cytolytic activity (i.e., generation of cytotoxic T cells specific for an immunogen). For CD4+ T cells, a preferred method for detecting specific T cell activation is the detection of the proliferation of T cells. For CD8+ T cells, a preferred method for detecting specific T cell activation is the detection of the generation of cytolytic activity.

Detection of the proliferation of T cells may be accomplished by a variety of known techniques. For example, T cell proliferation can be detected by measuring the rate of DNA synthesis. T cells which have been stimulated to proliferate exhibit an increased rate of DNA synthesis. A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca2+ flux, or dye uptake, such as 3-(4,5-dimethylthiazol-2-yl)-2,5-diphenyltetrazolium. Alternatively, synthesis of lymphokines (e.g., interferon-gamma) can be measured or the relative number of T cells that can respond to an immunogen may be quantified.

In some embodiments, expression of interferon proteins can be used to demonstrate immune system activation. Because interferon proteins have pleiotropic effects on other cells, interferon expression can also be determined by assaying interferon function. For example, interferon's can induce expression of hundreds of genes, including PKR, PML, RAP46/Bag-1, phospholipidscramblase, 2-5A synthetase, indoleamine 2,3-dioxygenase, and IFN regulatory factors. Levels of the interferon induced gene products (e.g., mRNA and proteins) can be measured as described above to determine induction of interferon. Thus, induction of interferon can result in a detectable increase in expression of an interferon regulated gene. Expression of message or protein levels can be used to determine induction.

In addition, other downstream effects of interferon (e.g., inhibition or decrease in rate of proliferation or induction of apoptosis; effects on immune effector populations that interact with tumor cells; and from inhibition of angiogenesis) can also be assayed to determine interferon induction.

Methods of Immune System Activation by Novel TLR Ligands, Alone or in Combination with IMPDH Inhibitors

Newly synthesized compounds are tested initially for their abilities to activate murine splenocytes or bone marrow-derived macrophages (BMDM). BMDMs are prepared as described in Chu (Chu et al., Cell, 103:909-918 (2000)). Different doses of new compounds (0.1-100 μM) as well as positive controls (ISS-ODN, R-848, Pam3Cys) are added to BMDM and the supernatants were collected after 24 hours of stimulation. Induction of cytokines by these compounds are measured by ELISA (TNF-α, IL-12, and IFN-γ). The expression of cell surface activation antigens (CD80, CD40, MHC class II) is determined cytofluorometrically using specific antibodies, as described (Martin-Orozco et al., Int Immunol, 11:1111-1118 (1999)). The compounds that show immune cell activation are further tested using cells from MyD88-deficient mice. Some TLR-initiated cell activation has been shown to be dependent on MyD88 (Takeuchi et al., Microbes Infect, 4:887-895 (2002)). TLR7 induction of IRFs and interferon production is shown herein to be dependent on DNA-PK.

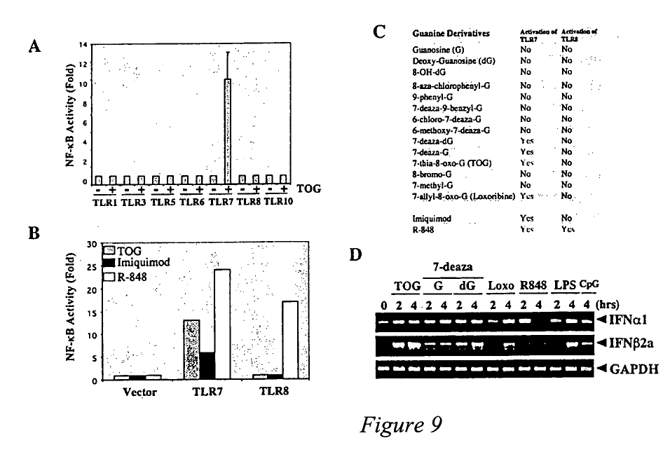

Compounds that activate immune cells via MyD88 or DNA-PK are screened for their respective receptors, using a transfection system described previously (FIG. 9A). Typically, 293-HEK cells are transfected using Lipofectamine with pCMV based vectors encoding individual TLRs, plus a NF-kB-Luciferase plasmid and a pCMV-β-galactosidase plasmid. After 24 hr of transfection, cells are stimulated with new TLR compounds for 6 hr, and luciferase activity of cell extracts are measured with a luminometer. The β-galactosidase activity is used for normalization as described (Chuang et al., J Leukoc Biol, 71:538-544 (2002)). Empty pCMV vector transfected cells are used for a negative control.

Human blood is obtained from the San Diego Blood Bank. Peripheral blood mononuclear cells (PBMC) are isolated from the blood samples using Ficollpaque. Mononuclear leukocytes are further purified by plating the interphase cells on collagen-coated tissue culture plates for 2 hr at 37° C. Adherent cells are detached, counted, and adjusted on 1× to 2×10⁶ cells/ml. The cells are stimulated with the different TLR ligands, alone and in combination. ELISAs for TNF-α and IFN-γ (Biosource International), and IFN-α and IFN-β (Research Diagnostics Inc.), are performed to measure the potency and maximal activities of the new compounds.

a) Synergism between TLR Ligands and IMPDH Inhibitors

IMPDH inhibitors such as ribavirin, mizoribine, and mizoribine base significantly enhance cytokine production from mouse bone marrow cells stimulated with TOG. IMPDH inhibitors are assayed in combination with known and newly synthesized activators of TLR7, TLR9, and TLR8. Cytokine production (interferon α, β, γ, TNF-α), both in murine BMDM, and human blood mononuclear leukocytes is assayed. The inhibitors are used at non-cytotoxic concentrations. Levels of Nuclear IRF-1, IRF-3 and IRF-7 increase markedly after exposure of bone marrow cells to TOG. A similar assay is used to determine the extent of activation of IRFs induced by TLR-L alone or in combination with IMPDH inhibitors. Nuclear and cytoplasmic fractions of mouse and human mononuclear cells are separated by centrifugation, electrophoresed, and immunoblotted with anti-IRF antibodies. Anti-tubulin antibodies serve as a control.

The experimental data is analyzed to determine both the potency and the maximal stimulatory capacity of the TLR7-L and TLR9-L, alone and in combination with a TLR7/8-L, and with various IMPDH inhibitors. The patterns of cytokine and costimulatory molecule production are compared. For each drug, and drug combination, an interferon (α, β, γ) to TNF ratio is calculated. Combinations that yielded the highest ratios are expected to show the best therapeutic to toxic ratio in vivo.

Microbial TLR-L are potent activators of innate immunity. Furthermore, TLR-activated DCs orchestrate adaptive immune responses to co-delivered antigens. All TLRs share common signaling pathways but vary in their expression on different subsets of DCs. Combinations of synthetic activators of TLR7, TLR9, and possibly TLR8, will potentially activate a broader spectrum of DCs and macrophages in vivo. Therefore, these combinations may have additive or synergistic immunostimulatory effects, especially when combined with IMPDH inhibitors. Various in vivo screening systems will be used to compare the effects of various synthetic TLR-L combinations on innate and adaptive immunity.

b) Screening System to Assess Synergism of Various TLR-L on Activation of Innate Immunity in vivo.

A single s.c. injection of ISS-ODN (50 μg per mouse) to BALB/c mice resulted in detectable serum levels of cytokines and the induction of co-stimulatory molecules expression which last up to three weeks post-injection (Kobayashi et al., Cell Immunol, 198:69-75 (1999); Martin-Orozco et al., Int Immunol, 11:1111-1118 (1999)). Interestingly, the induction of co-stimulatory molecules was in part the product of interferon (IFN) secretion. Other studies demonstrated similar protective kinetics from lethal Listeria challenge (Klinman et al., Infect Immun., 67:5658-63 (1999)). To explore the magnitude and the kinetics of the immunostimulatory effects of TLR-L in combination, BALB/c mice are injected s.c., once, at the nape of the neck, with the TLR-L, alone or in combination with IMPDH inhibitors. To assess the resulting activation of innate immunity, serum levels of cytokines, i.e., TNFα, IL-12, IFNα and IFNγ are assessed by ELISA and the level of expression of CD40, CD80 and CD86 are determined by FACS on F4/80+ (or CD 14+) splenocytes (macrophages) 1, 3, 7 14, 21, and 28 days after injection (4 mice per time point per TLR-L combination). The same combinations are delivered intranasally (i.n.), (Kobayashi et al., Cell Immunol, 198:69-75 (1999)). Cytokine levels are assessed in the serum and in the bronchoalveolar lavage fluid (BALF) by ELISA and the expression of co-stimulatory molecules on F4/80+ (or CD14+) cells is assessed by FACS on cells isolated from the bronchial lymph nodes and from the spleen (FACS). Similar analyses is performed on CD11c+ splenocytes (DCs).

Intra-gastric administration (by gavage) of ISS-ODN prevents the development of experimental colitis and mortality in mice (Rachmilewitz et al., Gastroenterology, 122:1428-1441 (2002)). This data also suggests that activation of innate immunity occurs in the GI tract by this route of administration. TLR-L combinations are administered by gavage. Cytokine levels are assessed in the serum (ELISA) and the expression of co-stimulatory molecules on F4/80+ (or CD14+) and CD11+ cells (FACS) is assessed on cells isolated from the spleen, Peyer's patches and lamina propria.

c) Assessment of Various TLR-L on Activation of Adaptive Immunity

In contrast to the immediate and non-specific nature of innate immunity, adaptive immunity is antigen-specific and results in immune memory. Some combinations of TLR-L will enhance maturation of DCs, which consequently will orchestrate CD4 and CD8 responses. A highly sensitive and reproducible in vitro screening system will provide a rapid means of assessing the ability of TLR-L, with or without IMPDH inhibitors, to enhance CD4+ and CD8+T cell activation. The most potent combinations are tested as adjuvants in vivo.

The capacities of various TLR-L combinations to induce maturation of DCs is first assessed in vitro. Murine bone marrow-derived DCs (BMDC) express all the known murine TLRs. This observation allows for the screening of murine DCs. Bone marrow from femurs and tibia of C57B1/6 mice are plated on day 0 into bacterial Petri dishes at 2×10⁵ cells/ml, in media containing 5 ng/ml of recombinant murine GM-CSF. The non-adherent cells are harvested on day 7, and analyzed by flow cytometry, after staining with antibodies against the following cell surface markers: CD11c (clone HL3), CD3 (clone 145-2C11), CD4 (clone RM4-5), CD8 (clone 53-6.7), CD11b (clone M1/70), CD14 (clone 2.4G2), CD40 (clone 3/23), CD54 (clone3E2), CD80 (clone 16-10A1), CD86 (clone GL1), H-2Kb (clone AF6-88.5), I-Ab (cloneM5/114.15.2), Gr1 (clone RB6-8C5), B220 (clone RA3-6B2), and NK1.1 (clone PK136, all from Pharmingen). Further purification of the cells is done using anti-CD 11c magnetic beads and a MACS column per manufacturer's instructions (Miltenyi Biotec) is performed for selected studies.

BMDC are treated with titrated concentrations of the above synthetic TLR-L and combinations thereof. The levels of IL-12, TNFα and IFNα in the supernatant are determined by ELISA. The up-regulation of co-stimulatory molecules (CD40, CD80 and CD86) and MHC class I and class II molecules is assessed by FACS 48 hrs post-stimulation. After identifying synergistic combinations of TLR-L, experiments are repeated in the presence of IMPDH inhibitors and the results compared to those obtained with the TLR-L combinations alone.

d) Assessment of CD4+ T Cell Activation in vitro

To test the ability of various TLR-L to enhance CD4+ T cell activation, BMDC (C57B1/6) are treated overnight with LPS-free HPLC-purified, chicken ovalbumin (OVA) and each TLR-L at concentrations found to activate DCs. CD4+ T cells from sex-matched OT2 mice that express a transgenic T cell receptor that recognizes MHC class II (I-A^{b})-restricted OVA (Barnden et al., Immunol Cell Biol, 76:34-40 (1998)) are purified from splenocytes using anti-CD4 magnetic beads. The purified CD4+ T cells are labeled with 1 μM carboxy-fluorescein diacetate, succinimidyl ester (CFSE), washed and co-incubated with an equal number of the CFSE-labeled CD4+ T cells in supplemented RPMI media for two days. Flow cytometry is done on the transgenic T cell population to assess T cell proliferation, as indicated by halving of CFSE fluorescence intensity in daughter cells produced with each round of proliferation. IFNγ, IL-13, IL-4, IL-5, and IL-10 secretion by the CD4+ cells is also assessed by ELISA. After identifying the CD4 activation profile per TLR-L, the various combinations are assayed with and without IMPDH inhibitors. Results are compared and those combinations that provide the optimal Th1 responses (high IFNγ and low IL-4, IL-5, IL-13) are further evaluated further in in vivo experiments.

e) Assessment of CD8 T Cell Activation in vitro

CD8+ T cells are usually activated when cytosolic antigen is presented to them on MHC class I by DCs in the context of CD4+ T cell helpers. Microbial TLR ligands enable DCs to cross-prime CD8+ T cells against exogenously acquired antigen (OVA) in a CD4+ T cell-independent fashion. This ability to elicit CD8+ T cell responses may help overcome the inability of current protein-based vaccines to elicit CTL responses that are important against intracellular infections. The ability of various TLR-Ls to enhance CD8+ T cell activation is assessed in a similar manner to that described for CD4 cells, except that purified CD8+ T cells from OT1 mice, which recognize MHC class I (H2K^{b})-restricted OVA (Hogquist et al., Cell, 76:17-27 (1994)), are used instead of CD4+ T cells. After identifying the CD8 activation profile per TLR-L (proliferation, cytokine production and CTL activity against EL-4 target cells pulsed with OVA derived MHC class I peptide), the various TLR-L combinations with IMPDH inhibitors are assayed. The results are compared, and those combinations that provide the highest CD8 responses (CTL activity and IFNγproduction), are further evaluated in in vivo experiments.

f) Activity of TLR-L Combinations in vivo

The studies presented above can be considered as an “in vitro immunization” and will provide insight into the adaptive immune responses elicited by various synthetic TLR-L in vivo. Assessment of their effects after systemic and mucosal immunizations are described below.

g) Dendritic Cell Maturation in vivo

The administration of a TLR9-L (i.e., ISS-ODN) pre-primed the immune system to a subsequent antigen challenge for at least two weeks (Kobayashi et al., Cell Immunol, 198:69-75 (1999)). The pre-priming period can be expanded by the administration of potent TLR-L/IMPDH inhibitor combinations. To assess the resulting DC maturation in vivo the TLR-L combinations identified above are injected once s.c to C57B1/6 mice (8 mice per group). Certain mice were killed 1, 3, 7, 14, 21, and 28 days after injection. The CD11c+ splenocytes are isolated and the expression levels of CD40, CD80, CD86, and MHC classes I and II are evaluated by FACS. Another set of DCs is incubated with OVA-derived, MHC class II peptide and then washed extensively, and the peptide pulsed DCs are incubated with equal number of CFSE-labeled CD4+ OT2 cells. The CD4 response (proliferation and cytokine profile) is assessed as described above. The same procedure is repeated with OVA derived, MHC class I pulsed DCs incubated with CFSE labeled CD8+ OT1 cells. The CD48 response (proliferation, CTL activity and cytokine profile) is assessed as described earlier.

Following a similar administration protocol, the same TLR-L/IMPDH inhibitor combinations are delivered i.n. (8 mice per group). OVA specific CD4 (OT2) and CD8 (OT1) responses are assessed with DCs isolated from the bronchial lymph nodes and from the spleen and pulsed with MHC class II and class I peptides, respectively, as described above.

To test the in vivo effects of the synthetic TLR-L/IMPDH inhibitor combination, identified above, C57B1/6 mice are immunized i.d at the tail base with the 4 most potent combinations identified above (4 mice per combination) and OVA (50 μg) on day 0 and day 14. Mice are given an intravenous boost of OVA (50 μg) 3 days prior to sacrifice on day 28. Serial retro-orbital bleeds will be done and OVA-specific antibody responses (IgG1, IgG2a and IgA) are determined by ELISA. OVA specific cytokine (IFN-γ, IL-13, IL-4, IL-5, and IL-10) and CTL responses are assessed using CD4+ and CD8+ splenocytes, respectively.

ISS-ODN is a potent mucosal adjuvant for HIV related antigens (Horner et al., J Immunol, 167:1484-1591 (2001)). To assess mucosal immune responses, i.n administration of OVA mixed with the 4 most potent TLR-L/IMPDH inhibitor combinations identified above (4 mice per combination) are done as described above for i.d immunization. Antibody profile (serum and BALF levels of IgG1, IgG2a and IgA), antigen specific cytokine profile of splenocytes and bronchial lymph node cells as well as CTL responses in the spleen and in bronchial lymph nodes are assessed. Similar studies are performed with intra-gastric administration. The immune responses are assessed at systemic (spleen) and at mucosal sites (lamina propria and Peyer's patches) as described (Horner et al., J Immunol, 167:1484-1591 (2001)).

The immune parameters described above were selected as surrogate markers of protection. Additional investigations can be performed to identify the secretion of other cytokines or chemokines and the induction of other co-stimulatory receptors on DCs as well as the memory profile of activated CD4 or CD8 cells. However, it is anticipated that many of the tested TLR-L will induce immune responses and that there will be a correlation between responses seen in vitro and in vivo. Identification of TLR-L or combinations of TLR-L and IMPDH inhibitors that induce the most potent immune responses are further explored to test their protective effects on lethal infection, as described in the next section. TLR-L that are potent inducers of innate immunity will have potential use as an early intervention against a wide variety of pathogens with the aim of preventing infection (with or without antibiotics) and/or progression of disease. TLR-L combinations that are identified as potent inducers of adaptive immunity, which may or may not be the same combinations that induce the best innate immune responses, can be used as adjuvants in vaccines against specific bio-terrorism grade pathogens.

Methods of Protecting a Mammal from Bacterial and Viral Infections by Administration of Novel TLR Ligands, Alone or in Combination with IMPDH Inhibitors.

a) Introduction

Promising TLR-L combinations identified as described above are tested against actual pathogens, in vitro and in vivo. Initially, the activities of TLR-L are tested individually, in combination, and with IMPDH inhibitors, using in vitro models of Salmonella and Listeria infections. These studies are based on, and will complement, the experiments described in the previous sections. Both the cytokine inducing activity and the ability to stimulate antimicrobial activity are determined for each compound. The results guide the selection of combinations to be used in in vivo mouse protection experiments. The animal studies ascertain the in vivo activities of TLR-L/IMPDH inhibitor combinations against the bacterial pathogens, and against representative viral infections, the Coronavirus that causes SARS, Punta toro and cowpox.

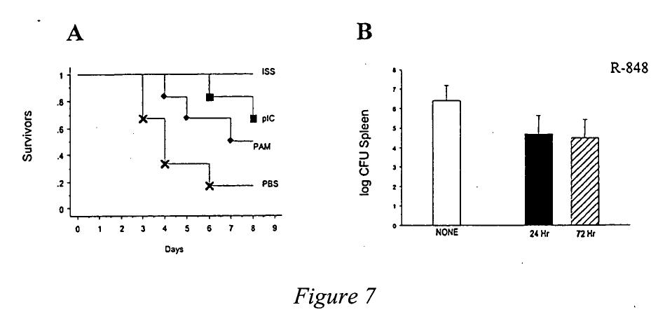

By definition, a successful pathogen is able to overcome the innate immune response. Innate immune responses can be made more effective by using TLR-L/IMPDH inhibitor combinations that are more effective than natural ligands in the activation of innate immunity. Indeed, pre-treatment of animals with some TLR-L such as LPS and poly I:C protects animals from some bacterial and viral infections (Smee et al., Can J Infect Dis 3(suppl B): 41B-48B (1992); Frey et al., J Bacteriol, 170:2078-2082 (1988)). Unfortunately, these compounds are too toxic to be of clinical use. In contrast, the injection of a TLR9-L, ISS-ODN, protects mice from Mycobacterium avium infection (Hayashi et al., Infect Immun, 69:6156-6164 (2001)), Francisella tularensis (Elkins et al., J Immunol, 162:2291-2298 (1999)), Listeria monocytogenes (L.m.) (Krieg et al., J Immunol, 161:2428-2434 (1998)), and M. tuberculosis (Juffermans et al., Infect Immun, 70:147-152 (2002)). Furthermore, the synthetic TLR7/8-L, R-848, protects mice against Mycobacterium bovis and Leishmania major infections (Buates et al., J Infect Dis, 179:1485-1494 (1999); Moisan et al., Antimicrob Agents Chemother, 45:3059-3064 (2001)). The protective effect of ISS-ODN against Listeria infection in BALB/c mice has been confirmed, and other TLR-L are also protective (FIG. 7).

b) Experimental Design

(1) Toxicity. TLR-Ls are tested for safety by administering varying doses each day×7 from 0.1 mg/kg to 100 mg/kg, which includes the dose range that has been tested for R-848 (a TLR7/8-L) and shown to be safe in mice (Vasilakos et al., Cell Immunol, 204:64-74 (2000)). Weight loss and lethality are determined after i.p. injection, and subsequently after p.o. and i.n. administration. Toxicity measurements include serum hemoglobin, ALT, AST, bilirubin, BUN and creatinine. Selected drug combinations are tested for toxicity, using dosages and routes of administration determined as described above.

(ii) Antibacterial activities. Promising TLR-L, alone and in combination with IMPDH inhibitors (i.e., basically those giving a high IFN/TNF ratio) are produced and tested in animal and in vitro models of infection. TLR9-L and TLR7-L are injected sub cutaneously. 10 μg of ISS-ODN (the dose that protects mice against Listeria monocytogenes) is tested against Salmonella enterica serovar Typhimurium 14028 nalr, hereafter referred to as S. typhimurium. BALB/c.D2 Nrampl congenic mice are infected orally with S. typhimurium in 0.1 M NaHCO3 (Heffeman et al., J Infect Dis, 155:1254-1259 (1987)). ISS-ODN is administered 1, 3, 10, and 30 days before infection, to get an estimate of the time needed to induce protection and the duration of that effect. Feces are collected 8 and 24 hours after infection and plated quantitatively on XLD with naladixic acid to see if the TLR-L reduces gut colonization. Six treated and control mice (recipients of non-stimulatory DNA) are sacrificed at 24 hours after infection in order to perform quantitative cultures of the distal three Peyer's patches, ileal wall, mesenteric lymph nodes, and the liver and spleen as previously described (Heffeman et al., J. Infect Dis, 155:1254-1259 (1987)). Since mice do not develop diarrhea after oral infection with Salmonella, colony counts in gut tissue and feces are used as an indication of whether the TLR-Ls are effective at reducing the severity of gut infection 4 days after infection. 10 treated and control mice are sacrificed to culture livers and spleens. Although B/c.D2 mice do not die from oral infection, the organism normally spreads to the liver and spleen by day 4 after infection. The percentage of mice with positive cultures of the livers and spleens is determined, and the geometric mean value of those colony counts. Similar studies are performed with other TLR-L, their combinations, and with IMPDH inhibitors. The optimal combinations are selected for further studies.

The experiments outlined above are repeated after mucosal administration (p.o. and i.n.) of the TLR-L and IMPDH inhibitors. Mice are challenged orally and the severity of infection is evaluated as above. The efficacy of mucosal to systemic administration in the inhibition of local invasion of Salmonella into gut tissues is compared.

Because it is unreasonable to expect that TLR-L or their combinations will be sufficient to combat Salmonella infection, the agents are tested in concert with a potent antimicrobial, ciprofloxacin. In these experiments nalr S. typhimurium is not used, as that organism has reduced sensitivity to the fluoroquinolone. Ciprofloxacin is administered by gavage 12 hours after infection b.i.d. for one day and mice are sacrificed 12 hours later for quantitative bacterial counts in the tissues listed above. The following treatments are assessed: TLR-L, TLR-L and IMPDH inhibitor, ciprofloxacin, ciprofloxacin+TLR-L, ciprofloxacin+TLR-L and IMPDH inhibitor and untreated controls (estimate 10 mice per group).

The second animal model is listeriosis in mice in which systemic administration of ISS-ODN was shown to be protective from i.p. or i.v. lethal infection (Krieg et al., J Immunol, 161:2428-2434(1998); Elkins et al., J Immunol, 162:2291-2298(1999)). Since Listeria monocytogenes normally enter via the gut, the ability of the TLR-L combinations to prevent or reduce systemic spread of oral infection is studied. Mouse intestinal epithelial cells do not express an E cadherin that binds to listerial internalin. However, transgenic mice expressing human E cadherin are a good model for the intestinal phase of the infection (Lecuit et al., Science, 292:1722-1725 (2001)). The smallest dose of strain oral Listeria monocytogenes 104035 that results in reproducible infections is determined and used as the inoculum in these mice. The mesenteric nodes and the small intestine are cultured quantitatively 24 and 48 hours after infection comparing the various TLR-L/IMPDH inhibitor combinations delivered s.c. Since the systemic spread of Listeria kills people, reduction of systemic invasion and growth of Listeria is evaluated after administration of TLR-L. Thus, mice are orally infected with a dose that is ˜2 LD50 and livers and spleens are cultured 24, 48, and 72 hours after infection. The same treatment is evaluated by mucosal administration (i.n. and p.o.). TLR-L combination and the routes of administration are also tested with oral amoxacillin, an effective treatment for Listeria that relies on host defenses to be optimal. Geometric mean colony counts will be determined for each organ.

(iii) Anti-viral activities. Viral infections are studied including The Coronavirus that causes SARS, Punta toro (PTV) and cowpox. PTV is a Flavivirus in the Bunyavirus family that is closely related to Rift Valley Fever virus, and it is quite sensitive to type 1 interferons. Ten gram C57/BL6 (B6) mice are infected sub-cutaneously with 10⁶ plaque-forming Units (PFU) of the Adames strain of PTV grown in Monkey kidney cells, a dose that kills 90% of weanling mice (Sidwell et al., Ann NY Acad Sci, 653:344-355 (1992)). 20 placebo treated controls and 10 mice each are treated with a TLR7/9 ligand. Each dose is tested with and without a dose of an IMPDH inhibitor, which will potentiate TLR mediated signaling. A range of doses and schedules of TLR ligands are tested, based on toxicity studies. One group of 10 mice receives only the IMPDH inhibitor. Mice are followed for survival for 21 days. The most effective dose combination is chosen and the experiment repeated, but three mice in each group are sacrificed on day 5 after infection for quantitative viral cultures of the liver. Liver discoloration is scored on a 1-4 scale, and serum is obtained for measurement of ALT and circulating interferons by ELISA. The virus causes hepatitis in mice and the reduction in liver inflammation as assessed by discoloration and ALT levels is another measure of effective treatment of PTV. In this model and both TOG and ribavirin have significant activity (Sidwell et al., Can J Infect Dis 3(Suppl B):49B-54B (1992); Smee et al., Antiviral Chem Chemother, 2:93-97 (1991)). Kaplan-Meier survival curves are plotted and significant differences in mortality rates determined using the Mann-Whitney U test. The geometric mean viral titers and the mean score for ALT are compared using the t test; the liver discoloration score is compared using Wilcoxon-Rank Sum analysis.

Cowpox is a rodent Orthopoxvirus that infects cows. This group of viruses includes Variola (smallpox), and they are structurally similar and use the same replication strategy. These viruses express a set of soluble secreted decoy cytokine receptors that are responsible in part for their resistance to IFN (Smith et al., J Gen Virol, 81:1223-1230 (2000)). Mice are infected intra-nasally with the Brighton strain of cowpox propagated in African green monkey cells as described (Smee et al., Antiviral Res, 54:113-120 (2002)). Mice are anesthetized with ketamine prior to inoculating 5×10⁵ PFU. Mice are pretreated with the TLR-L or combination chosen as above and administered either s.c., or i.n. Untreated mice are controls, and uninfected mice are controls for toxicity of the ligand. The ability of the TLR-L to potentiate the antiviral activity of cidofovir (30 mg/kg i.p.) is also tested, beginning the day after infection and continuing for 2 days. TLR-L is also tested as a treatment for the virus after infection alone and in combination with IMPDH inhibitors. On day 6 the mice are sacrificed and lung viral titers are determined. IMPDH inhibitors are also tested alone for ability to treat the virus. Cidofovir at this dose protects all mice from death but they still have ˜10⁷ PFU/g of lung (Smee et al., Antiviral Res, 54:113-120 (2002)).

Mice are infected intra-nasally with the Coronavirus that cause SARS. Mice are anesthetized with ketamine prior to inoculating 5×10⁵ PFU. Mice are pretreated with the TLR-L or combination chosen as above and administered either s.c., or i.n. Untreated mice are controls, and uninfected mice are controls for toxicity of the ligand. The ability of the TLR-L to treat the virus after infection alone and in combination with IMPDH inhibitors is also tested. IMPDH inhibitors are also tested alone for ability to treat the virus. On day 6 the mice are sacrificed and lung viral titers are determined.

(iv) Effect of TLR-L combinations on protective immunity against antigens associated with infectious agents. Adaptive immune responses against L. monocytogenes in mice are assessed. Previous studies with this bacteria identified that the CTL response is crucial for protective immunity. Heat killed preparations of L. monocytogenes (HKLM) do not protect from lethal challenge, but prime CTL responses (Lecuit et al., Science, 292:1722-1725 (2001)). Thus, BALB/c mice (6 mice per group) are immunized intradermally with 10⁹ HKLM, the most potent TLR-L combinations identified for the model antigen OVA, or with HKLM with the TLR-L combinations at day 0 and 14 (10 mice per group). Eight weeks after the last immunization, mice are challenged i.v. with a lethal dose of 10⁵ CFU of bacteria and survival of these mice is assessed during the two week period post-challenge. Additional groups are killed three and seven days post-infection and colony counts are assessed in the liver and spleen (6 mice per group). The role of CD4 and CD8 in mediating the protection against lethal LM challenge is evaluated by injection of anti-CD4 or anti-CD8 antibodies to the immunized mice i.v. two days prior to lethal challenge. Mice are also immunized i.n. with the human E cadherin Tg mice (Lecuit et al., Science, 292:1722-1725 (2001)) with HKLM and combinations of TLR-L and challenge the immunized mice orally with the lethal infectious dose identified above. Those of skill will recognize that adaptive immune responses against other infectious pathogens can be assayed in a similar manner.

(v) Studies with human PBMC. As discussed earlier, (a) human but not mouse macrophages express TLR8, whereas (b) mouse but not human macrophages express TLR7 and TLR9. Two different approaches are used to study the effects of various TLR7, 8, 9-L combinations, with or without IMPDH inhibitors, on the antimicrobial activity of human macrophages and DCs. In the first method, the direct effect of TLR-L on human macrophages is determined. In the second approach, mixed effector cell populations are used to produce cytokines that will activate macrophages and augment the antimicrobial response. This second method closely models the in vivo situation in which a variety of host cell types are affected by TLR-L.

In the first series of experiments, purified macrophage populations are stimulated with the TLR-L combinations. The optimal combinations are determined, in part, by the activity of these combinations in inducing cytokine production and protection from lethal infections as determined above. However, a range of agents are tested in dose response studies, and combination analyses as well. For macrophage systems, primary human monocyte-derived macrophages are used. Human monocyte-derived macrophages are isolated from PBMCs by differential adherence as described and allowed to differentiate 3 days (Libby et al., Cell Microbiol, 2:49-58 (2000)). These cultures may also contain small numbers of DCs. Macrophages are treated with the TLR-L or combinations for 24-72 hours, then infected with L. monocytogenes or S. typhimurium. In both cases, the multiplicity of infection is approximately one bacterium per macrophage. This inoculum allows for substantial growth of the bacteria in non-treated macrophages. Listeria uptake is efficient without opsonization, but Salmonella require opsonization with 50% normal autologous serum for reliable results. The efficiency of phagocytosis is measured by comparing the inoculum with the bacteria recovered after 30 minutes of phagocytosis and one hour of gentamicin treatment to kill the extracellular bacteria (Libby et al., Cell Microbiol, 2:49-58 (2000)). Viable bacteria are determined by measuring CFU at 6, 12, and 24 hours after infection. The positive control for antimicrobial activity is cells treated overnight with IFN-γ (100 ng/ml). Listeria grows in untreated human macrophages under these conditions, increasing 10-fold or more after 6-24 hours depending on the cell type. IFN-γ severely inhibits this growth in both murine and human cells. Salmonella proliferate in untreated human macrophages, resulting in cytotoxicity manifested by detachment of macrophages, easily detected by collecting the culture supernatant (Libby et al., Cell Microbiol, 2:49-58 (2000)). IFN-γ not only blocks bacterial growth, but induces profound bactericidal activity with killing of 90-99% of the inoculum over 24 hours. Thus, the assay systems in all cell types produce a robust difference between positive and negative controls for antimicrobial activity. These studies determine whether the TLR-L, or their combinations, will induce antimicrobial activity in isolated macrophage cultures. In addition, the ability of IMPDH inhibitors to potentiate the effects of TLR-L combinations is tested. TLR-L-stimulated macrophages are assayed for the respiratory burst and for NOS both before and following bacterial infection, as described previously (Sly et al., Infect Immun, 70:5312-5315 (2002))

It is likely that in vivo TLR-L interact with several cell types. In fact, much of the antimicrobial activity for macrophages generated in vivo may be due to cytokine production by non-macrophage cell types. As a model of this situation, human PBMCs are exposed to the TLR-L in vitro. In humans, DC subsets express different levels of TLR 7 and 9, with differential cytokine responses to these ligands (Ito et al., J Exp Med, 195:1507-1512 (2002)). The use of PBMCs ensures that both plasmacytoid and myeloid DC subsets are represented in the assay system. After 24-72 hours, the culture supernatants from these cells are harvested and added to murine or human macrophages respectively, with or without added TLR-L. In addition, the effects of IMPDH inhibitors are also tested in this system. After 24 hours of treatment, these macrophages will be infected with Listeria and Salmonella as described above, and the effects of the culture supernatants on phagocytosis and bacterial growth are determined.

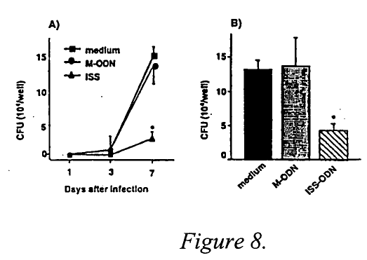

Another potential use of TLR-L is to synergize with antibiotic treatment of infections. For example, ISS-ODN acts synergistically with antibiotics on M. avium in macrophages (FIG. 8). Macrophage infections with Listeria and Salmonella are also studied. For Listeria, both ampicillin and ciprofloxacin have only a modest antibacterial effect on intracellular organisms at achievable serum levels (Carryn et al., Antimicrob Ag Chemother, 46:2095-2103 (2002)). Therefore, the effects of these antibiotics, alone and in combination with TLR-L that have some antimicrobial activity in the macrophage assay, are tested in macrophages infected with Listeria. For Salmonella, ciprofloxacin is an effective antibiotic, sub-inhibitory dosages are used together with TLR-L to detect any potential synergistic activity. In addition, synergistic effects of antibiotics, TLR-L, and IMPDH inhibitors are determined.

The materials, methods, and devices of the present invention are further illustrated by the examples which follow. These examples are offered to illustrate, but not to limit, the claimed invention.

### EXAMPLES

### Example 1

### Adjuvant Effects of ISS-ODN

1.1 Immunization with ISS-ODN

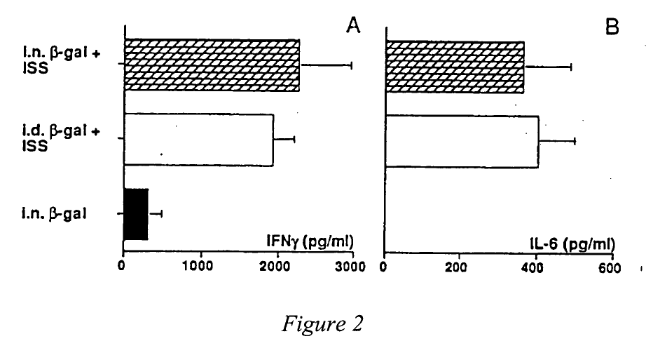

ISS-ODNs containing CpG motifs are specific TLR9 activators (Hemmi H et al., Nature, 408:740-745 (2000)). In this experiment, the relationship between immunization of mice with ISS-OSN and production of an immune response was examined. (FIG. 2) Phosphorothioate ISS-ODN 5′-TGACTGTGAACGTTCGAGATGA-3′, and a control mutated ODN (m-ODN) 5′-TGACTGTGAAGGTTAGAGATGA-3′ that lacks ISS activity, were used as prototypes.

1.1.a Methods

Female BALB/c mice received three immunizations with β-gal (50 ug) alone or with ISS-ODN (50 μg) 7 days apart, via the intranasal or intradermal route (FIG. 2). Splenocytes were harvested from sacrificed mice during week 7 and cultured in medium with or without β-gal (10 ug/ml), and ELISAs were assayed on 72-h supernatants. Antigen-induced splenocyte cytokine profiles are shown in FIG. 2. The profiles represent the mean of four mice in each group and similar results were obtained in two other independent experiments. Error bars reflect standard errors of the means (A) IFN-γ. (B) IL-6.

1.1.b Results

In mice, both intranasal and intradermal immunization with ISS-ODN, together with an exogenous antigen (either β-galactosidase or ovalbumin), stimulated serum Ig and lymphocyte cytokine responses with an equivalent Th1 bias (FIG. 2) (Horner A et al., Curr Top Microbial Immunol, 247:185-198 (2000)). Splenocytes from immunized mice cultured without β-gal produced negligible amounts of cytokines.

1.2 Time Lapse Before ISS-ODN Immunization

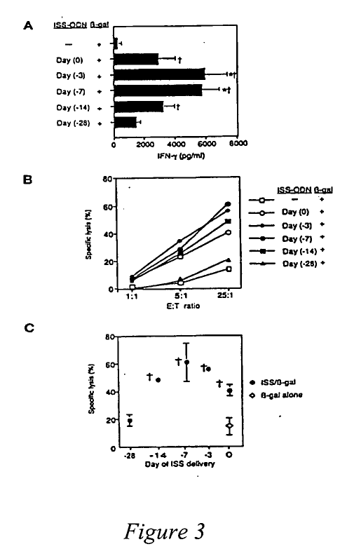

In this experiment, the amount of time between which a mouse exposed to an antigen can produce an immune response was examined. (FIG. 3)

The results in FIG. 3 represent the mean ± SE for four mice in each group. Similar results were obtained in two other independent experiments. Mice immunized with m-ODN either prior to or with β-gal immunization did not demonstrate an increased IFN-γ or CTL response when compared to mice immunized with β-gal along (data not shown). (A) IFN-γ response. Similar findings were observed for murine (i.n.) pre-priming (R). Mice receiving ISS up to 14 days prior to β-gal demonstrated an improved IFN-γ response when compared to mice immunized with γ-gal alone (†P≦0.05). Delivery of ISS from 3-7 days before β-gal led to an increased IFN-γ response when compared to mice receiving ISS/β-gal co-immunization (*P≦0.05) (B) CTL response. (C) Comparison of CTL response at an effector:target ratio of 25:1. Mice receiving ISS up to 14 days prior to β-gal demonstrated an improved CTL response when compared to mice immunized with β-gal alone (†P≦0.05).

The ISS-ODN could be administered up to two weeks before antigen exposure, and still potentiate both cellular and humoral immune responses (Kobayashi et al., Cell Immunol, 198:69-75 (1999)). This effect is known as “pre-priming”.

### Example 2

### TLR Ligands and Cytokine Production

2.1 Cytokine Responses

The cytokine responses of murine bone marrow mononuclear cells were compared to a TLR2-L (PGS), a TLR3-L (polyI:C), a TLR4-L(LPS), and a TLR9-L (ISS-ODN). IL-12, IL-10, IL-6, IL-1b, and KC (a chemokine) were assayed by ELISA (See 2.1.b Results). Notably, the ISS-ODN induced relatively more IL-12, and less IL-10, that did the other TLR activators.

2.1.a Methods

Bone marrow cells were cultured in triplicate at 5×10⁵ cells/ml with each TLR ligand in dose response studies covering a 3 log concentration range. The cells were cultured for 48 hours and supernatant cytokine levels were analyzed by ELISA. The minimal optimal concentration for each reagent was reported with a three fold increase in TLR ligand concentration not leading to any significant increase in cytokine production. Results are reported as means ± standard errors. n.d.=none detected

2.1.b Results

| TLR ligand | IL-12 (pg/ml) | IL-10 (pg/ml) | IL-6 (pg/ml) | IL-1b (pg/ml) | KC (pg/ml) |
|---|---|---|---|---|---|
| None | n.d. | n.d. | n.d. | n.d. | n.d. |
| PGS (100 μg/ml) | n.d. | n.d. | 976 ± 239 | n.d. | 2210 ± 149 |
| PolyI:C (100 μg/ml) | 570 ± 62 | 1374 ± 237 | 2380 ± 657 | 148 ± 27 | 349 ± 62 |
| LPS (1 μg/ml) | 3461 ± 437 | 1903 ± 438 | 5321 ± 1657 | 458 ± 53 | 644 ± 75 |
| ISS-ODN (10 μg/ml) | 15083 ± 1127 | 817 ± 342 | 443 ± 147 | 461 ± 127 | 545 ± 327 |

2.2 Systemic Adjuvants

Recently, the systemic adjuvant activities of other TLR-Ls were evaluated. In addition to ISS-ODN, PGS, LPS and polyI:C were found to be effective systemic adjuvants for vaccination, but they induced much less interferon-γ (IFNγ) than did ISS-ODN.

2.2.a Methods

Six-week-old BALB/c mice (4 per group) were intradermally immunized with ovalbumin (OVA) (20 ug) and TLR ligand on day 0 and day 7. During week 6, serum was collected and splenocytes were harvested and cultured with OVA (50 ug/ml). After 3 days, splenocyte culture supernatants were collected. Sera Ig levels and splenocyte supernatant cytokine levels were determined by ELISA. Results are reported as means ± standard errors. n.d.=none detected.

| Antigen | TLR ligand | IgE (U/ml) | IgGl (U/ml) | IgG2a (U/ml) | IFNg (pg/ml) | IL-5 (pg/ml) |
|---|---|---|---|---|---|---|
| OVA | None | n.d. | 1104 ± 627 | n.d. | n.d. | 41 ± 32 |
| OVA | PGS (50 ug) | 267 ± 29 | 3004 ± 258 | n.d. | n.d. | 154 ± 51 |
| OVA | PolyI:C (50 ug) | n.d. | 18853 ± 2439 | 2642 ± 380 | 672 ± 322 | 236 ± 87 |
| OVA | LPS (20 ug) | n.d. | 5771 ± 3254 | 2154 ± 628 | 320 ± 209 | n.d. |
| OVA | ISS-ODN (50 ug) | n.d. | 356 ± 186 | 12720 + 3020 | 6466 ± 4293 | 38 ± 38 |

### Example 3

### Antiviral Effects of ISS-ODN

3.1 Effect of ISS on RSV Replication in the Lung

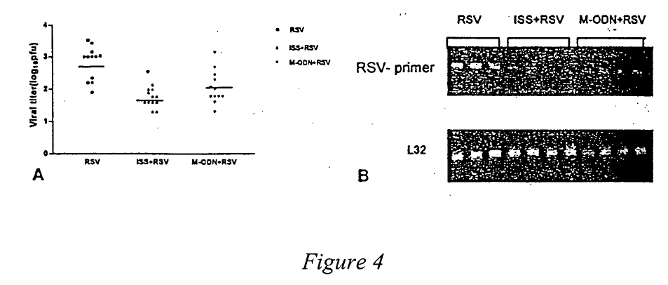

The effects of ISS on RSV replication in the lung was investigated (FIG. 4) (Cho et al., J Allergy Clin Immunol, 108:697-702 (2001)).

3.1.a Methods

Mice were pretreated with ISS-ODN (50 μg i.p.) six days before RSV infection (10⁶ pfu). Lung indices of RSV viral load were assessed 4-6 days after RSV infection.

3.1.b Results

ISS inhibits RSV replication in the lung. FIG. 4A demonstrates that ISS inhibits the number of RSV plaque-forming units (log 10 scale) in lungs of mice infected with RSV and treated with ISS compared with that seen in mice infected with RSV and treated with M-ODN (n=3; *P<0.001). Similarly, FIG. 4B demonstrates, by means of RT-PCR, that ISS inhibits the level of expression of the RSV-N gene in the lungs of mice infected with RSV and treated with ISS compared with that seen in mice infected with RSV and treated with M-ODN. Control housekeeping gene L32 expression is also depicted.

3.2 Effect of ISS on RSV-induced Peribronchial Inflammation

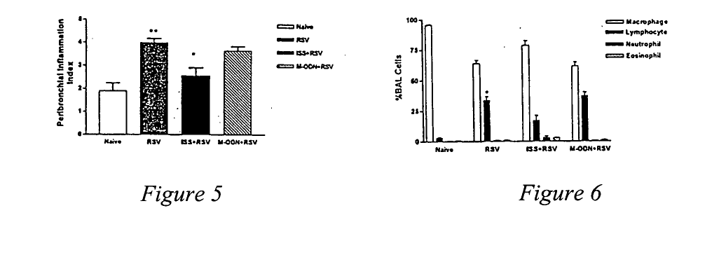

The effects of ISS on RSV-induced peribronchial inflammation was investigated (FIG. 5) (Cho et al., J Allergy Clin Immunol, 108:697-702 (2001)).

3.2.a Methods

Mice were pretreated with ISS-ODN (50 μg i.p.) six days before RSV infection (10⁶ pfu). Lung indices of RSV-induced bronchial inflammation were assessed 4-6 days after RSV infection.

3.2.b Results

RSV infection induced the expression of significant numbers of peribronchial inflammatory cells compared with that seen in uninfected mice (n=3; **P<0.05). ISS significantly inhibited the number of peribronchial inflammatory cells in the airways of RSV-infected mice treated with ISS compared with RSV-infected mice that had not received ISS (n=3; *P<0.05).

3.3 Effect of ISS on RSV-induced BAL Inflammation

The effects of ISS on RSV-induced BAL inflammation was investigated (FIG. 6) (Cho et al., J Allergy Clin Immunol, 108:697-702 (2001)).

3.3.a Methods

Mice were pretreated with ISS-ODN (50 μg i.p.) six days before RSV infection (10⁶ pfu). Lung indices of RSV-induced BAL inflammation were assessed 4-6 days after RSV infection.

3.3.b Results

RSV infection induced a significant increase in BAL lymphocytes compared with that seen in uninfected mice (n=3; *P<0.05). ISS inhibited the RSV induced increase in BAL lymphocytes compared with that seen in RSV-infected mice that had not received ISS, but this did not reach statistical significance (n=3; P=0.07).

The results showed that ISS-ODN induced the expression of interferon-γ in the lung (not shown), significantly reduced RSV titers (FIG. 4), and reduced bronchial inflammation (FIGS. 5 and 6).

### Example 4

### Antibacterial Effects of TLR-L

4.1 Effect of TLR-Ls on Listeria infection in Mice

The effect of TLR-Ls on the ability of Listeria to infect mice was examined in this experiment.

4.1.a Methods

Groups of six mice were injected with 10 μg of either ISS-ODN, Pam3Cys, or polyI:C three days before infection with 7×10⁵ Listeria monocytogenes. The mice were observed for the nine days. 5 mice also each received 10 μg of R-848, 24 hours before infection. The animals were sacrificed 72 hours later to determine CFU/spleen.

4.1.b. Results

As shown in FIG. 7A, all the tested TLR ligands protected mice. Even Pam3Cys-treated mice lived longer than controls (p=<0.001). The TLR7/8-L R-848 lowered bacterial counts nearly two logs at both dosing intervals (FIG. 7B).

4.2 ISS-ODN Stimulation: Human Mononuclear Leukocytes

The effect of ISS-ODN on the antibacterial activity of bone marrow-derived macrophages (BMDM) was investigated. (FIG. 8A).

4.2.a Materials

BMDMs were treated with 10 μg/ml each of ISS-ODN, or m-ODN for three days prior to infection with Mycobacterium avium (Hayashi et al., Infect Immun, 69:6156-6164 (2001)). Then, the intracellular growth of the mycobacteria was assessed by a colony forming unit (CFU) assay on days 1, 3, and 7 after infection (FIG. 8, panel A). Each condition was tested in triplicate, and the results are expressed as means ± SD CFU per well. The results in FIG. 8A are representative of three experiments.

4.2.b Results

The ISS-ODN, but not the m-ODN, significantly diminished the CFU count. Other experiments showed that the antibacterial activities of the ISS-ODN were attributable, in part, to increased levels of interferon-inducible enzyme indoleamine dioxygenase (results not shown).

4.3 ISS-ODN Stimulation: Human Macrophages

The effect of ISS-ODN on the anti-mycobacterial properties of clarithromycin (CLA) in BMDMs was investigated. (FIG. 8, panel B).

4.3.a Materials

BMDMs were treated with ISS-ODN or m-ODN immediately after infection, and M-avium growth was assessed by the CFU assay 7 days post infection. Each condition was tested in triplicate, and the results are expressed as means ± SD CFU per well. The results in FIG. 8B are representative of three experiments.

4.3.b Methods

The combination of ISS-ODN and CLA was much more effective in eliminating the bacteria than either drug used alone. Thus, the TLR9 ligand ISS-ODN has antibacterial effects toward both Listeria monocytogenes and Mycobacteria avium, and ISS-ODN potentiates antibiotic action.

### Example 5

### Activation of TLR7 by Guanine Analogs

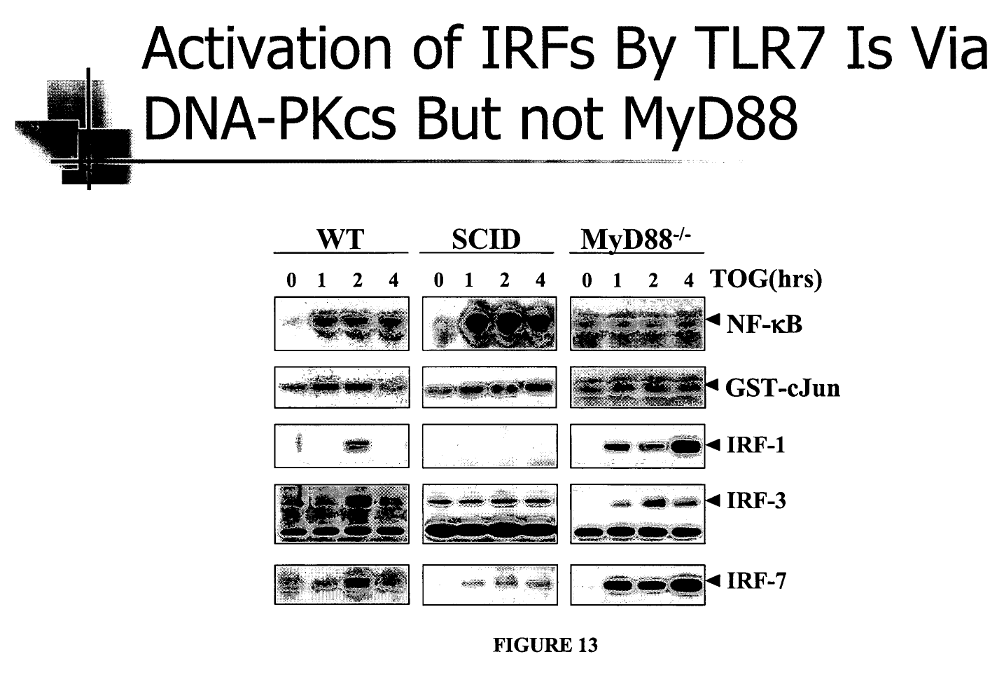

Several modified guanosine analogs have broad-spectrum antiviral activity in mice after parental or intranasal administration, but their mechanism of action was unknown. To address this issue, the 293 cell line was transiently transfected with vectors encoding TLR1-TLR10, or with empty vector as described (Chuang et al., J Leukoc Biol, 71:538-544 (2002)). Then, the cells were treated with the guanine analog 7-thia-8-oxoguanosine (TOG, FIG. 1). After six hours, NF kB activation was assessed using a reporter gene assay. Only the TLR7 transfected cells gave a positive signal (FIG. 9, Panel A). To confirm the specificity of the assay, TLR7 and TLR8 transfected cells were also incubated with the prototype TLR7-L imiquimod, and the TLR7 and TLR8 activator R-848 (Jurk et al., Nat Immunol, 3:499 (2002)). The results documented the specificity of the assay system and confirmed the TLR7 activating properties of TOG (FIG. 9, Panel B). Subsequently, 14 guanine-like compounds were assayed for TLR7 and TLR8 stimulation (FIG. 9, Panel C). Positive results were obtained with TOG, 7-deaza-2′-deoxyguanosine, 7-deazaguanosine, and 7-alkyl-8-oxo-guanosine. Activation of IRF's by TLR7 ligands appeared to depend on DNA-PK. (FIG. 13.) Other experiments showed that these guanine analogs induced type I interferon production by human blood mononuclear cells (FIG. 9, Panel D).

### Example 6

### Effect of Multivalency on the Potency of ISS-ODN

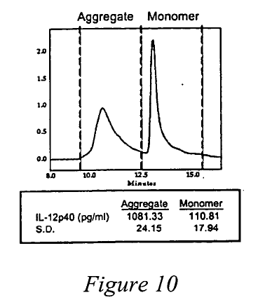

The multimerization of a ligand for a membrane bound receptor often increases its net avidity, and hence its ability to activate signal transduction. Guanine residues in ODN are known to promote aggregation, and when present in ISS-ODN enhance TLR9 activating activity (Lee et al., J Immunol, 165:3631-3639 (2000)). Therefore, aggregated ISS-ODN was separated from monomeric ISS-ODN by size exclusion HPLC. The specific activities of the two fractions were measured by IL-12 synthesis in mouse bone marrow cultures (FIG. 10). The aggregates had a 10-fold higher specific activity than did monomers with the same composition. By analogy, it is reasonable to assume that the incorporation of guanine or imiquimod TLR7 ligands into ODN-like molecules, alone or together with CpG motifs, would potentiate their immunostimulatory activity, by increasing their valence and/or by enhancing endocytosis.

### Example 7

### Potentiation of TLR Signaling by IMPDH Inhibitors

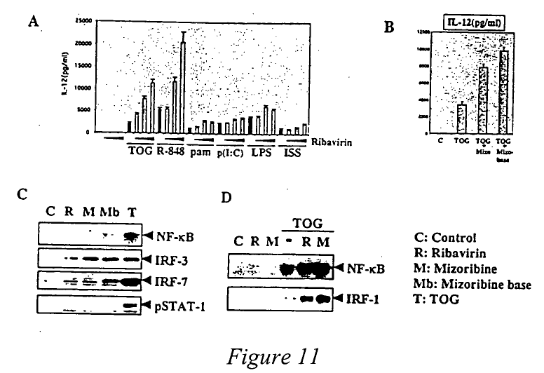

In mouse bone marrow derived macrophages, and in human peripheral blood mononuclear cells, the IMPDH inhibitors ribavirin, mizoribine, and mizoribine base, potentiated cytokine production induced by several different TLR-L (FIG. 11). The combination of IMPDH inhibitors and TOG or resiquimod (also known as R-848) had an effect greater than either compound on its own. (See, e.g., FIGS. 11A, C, and D.) Investigations of the molecular basis for the synergy revealed that the enzyme inhibitors on their own induced the phosphorylation and nuclear translocation of the key interferon regulatory factors IRF-1, IRF-3 and IRF-7. These effects were specific for macrophages and DCs, and were not observed in lymphocytes. Moreover, they were prevented in medium supplemented with guanine (not shown). Additional experiments in macrophages demonstrated that the enhancement of IL-6 production by R-848 in the presence of the IMPDH inhibitor ribavirin was also abolished by addition of guanosine to the media (not shown).

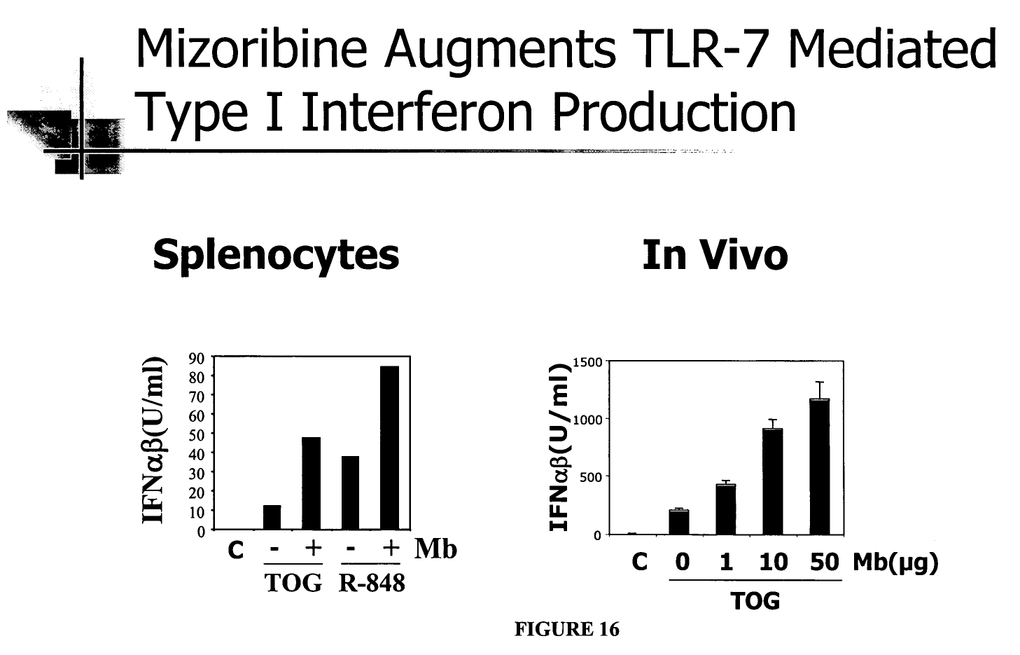

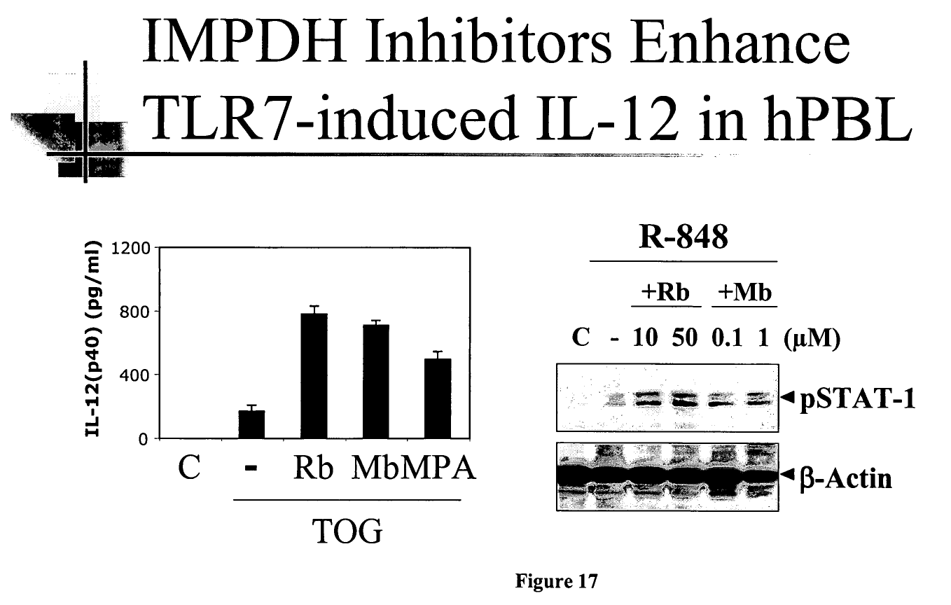

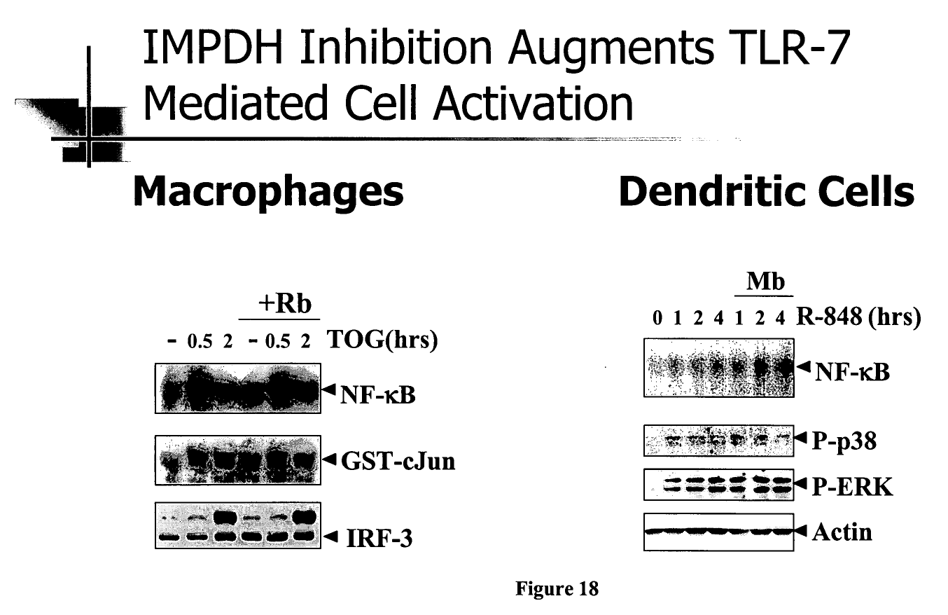

Mizoribine also augmented TLR7 mediated type I interferon production in spenocytes isolated from mice and in vivo in mice. Results are shown in FIG. 16. In splenocytes, production of interferon was increased more than two-fold by addition of either TOG or R-848. (FIG. 16, left panel.) In mice that were injected with 250 μg of TOG, addition of mizoribine increased levels of Type I interferon in bloodmore than 4-fold. Production of IL-12 was assayed in human peripheral blood leukocytes (hPBLs). Ribavirin (Rb), mizoribine base (Mb), and mycophenolic acid (MPA) each enhanced the increase of IL-12 production after stimulation with TOG. (FIG. 17, left panel.) Rb and Mb each enchanced activiation of the signal transducer and activator of transcription 1 (STAT-1) by TOG. (FIG. 17, right panel.) IMPDH inhibition also augments TLR-7 mediated activation in bone marrow derived macrophages or in bone marrow derived dendritic cells (DC's). (See, e.g., FIG. 18.)

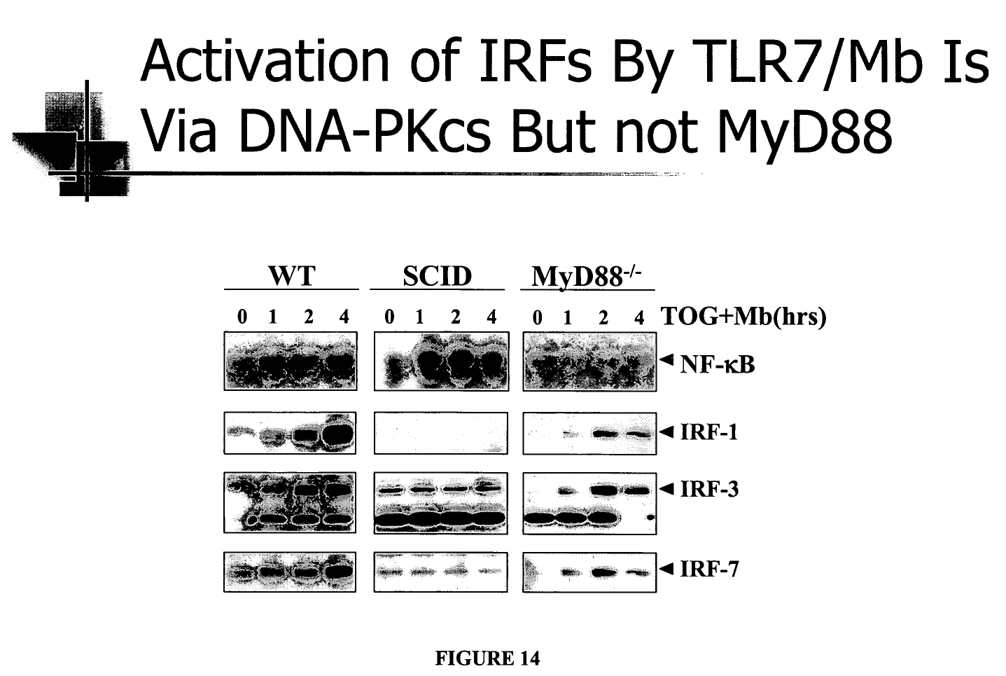

Activation of IRFs by IMPDH Inhibitors is dependent on DNA-dependent protein kinase (DNA-PK). Activation by Mb is shown. (FIG. 12.) Experiments were performed in cells from wild-type mice and SCID mice, which lack functional DNA-PK. Activation of IRFs by TLR7 and Mb is via DNA-PKcs but not MyD88. (FIG. 14.) Enhancement of TLR-mediated cytokine induction by IMPDH inhibitors is DNA-PKcs dependent. (FIG. 15.) The experiment was performed in cells from SCID mice.

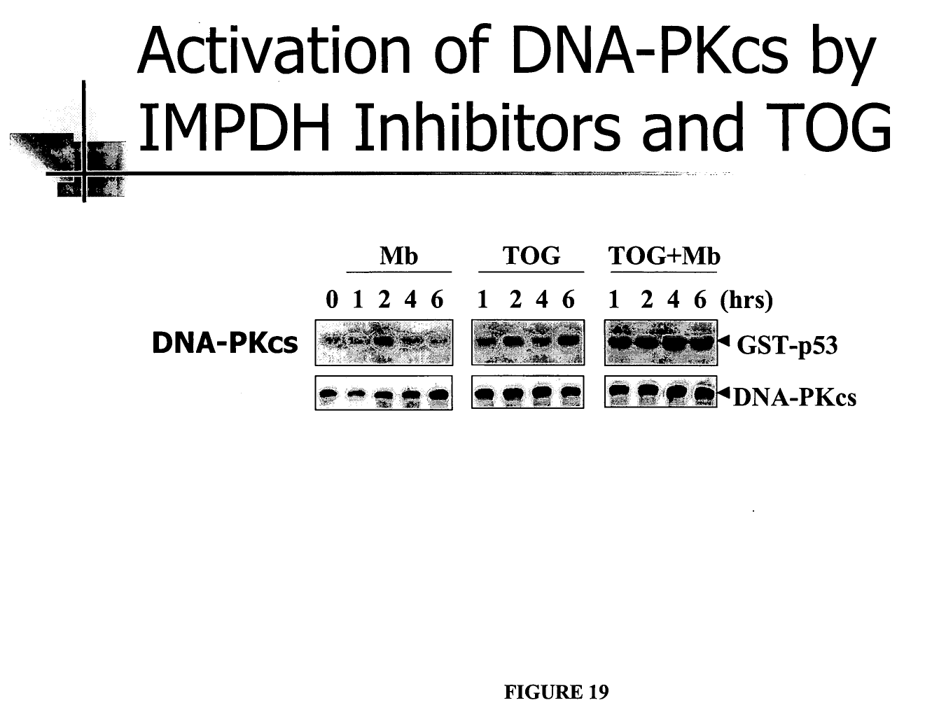

IMPDH inhibitors and TOG activate DNA dependent protein kinase catalytic subunit (DNA-PKcs). (FIG. 19.) BMDM were stimulated with the Mb alone, TOG alone, or a combination of Mb and TOG. Activation of DNA-PK was measured by in vitro kinase assay using a GST-p53 substrate or DNA-PKcs autophosphorylation. The combination of TOG and MB resulted in increased phosphorylation of substrates and earlier onset of phosphorylation.

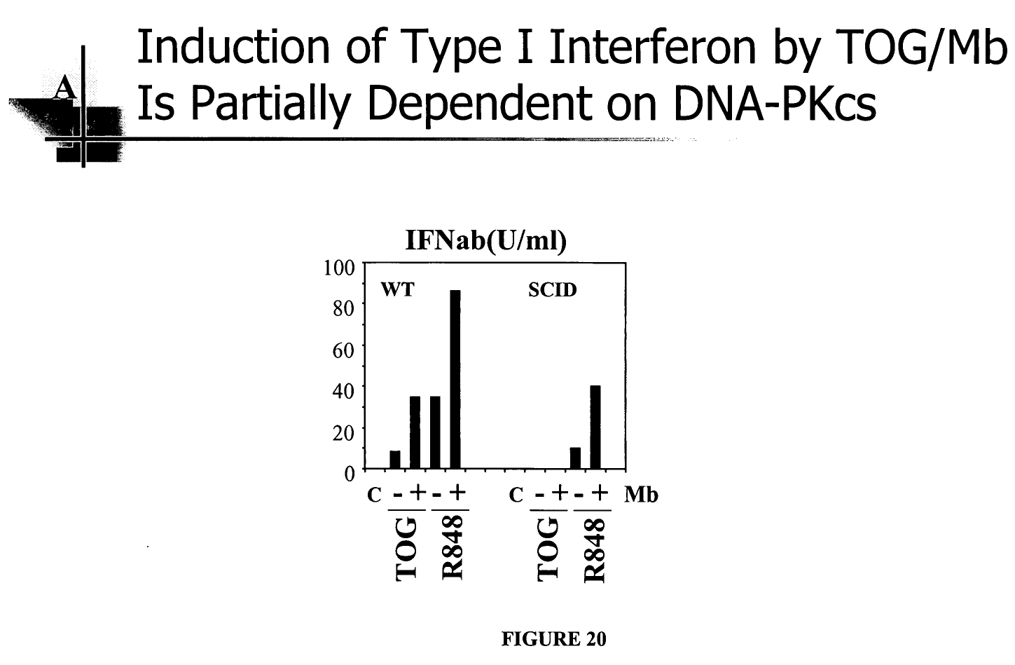

Induction of type I interferon by TOG combined with Mb is partially dependent on DNA-PKcs. (FIG. 20.) Splenocytes of WT or SCID mice were stimulated with the indicated stimuli and Type I interferon levels were determined by bioassay. Additions were in the following amounts: TOG (100 μM), R-848 (1 μM), and Mb (10 μM).

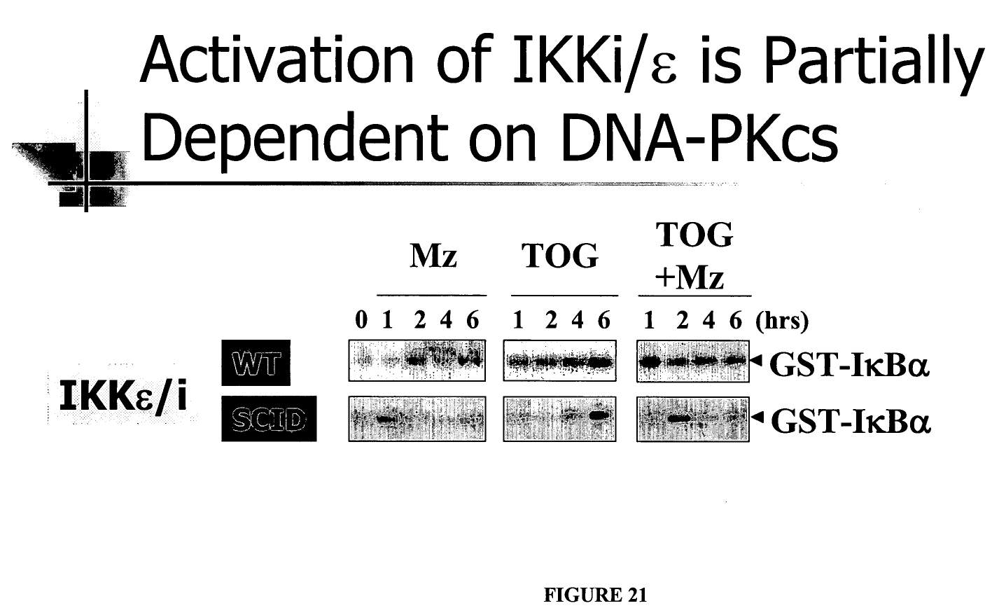

Activation of the kinase IKKi/ε is also partially dependent on DNA-PKcs. (FIG. 21.) BMDM of WT or SCID mice were stimulated with the indicated stimuli and activation of IKKi/ε was measured by in vitro kinase assay. GST IκBα was used as the substrate.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A compound having the formula: 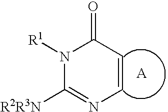
wherein
R¹, R² and R³ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl; and
ring system A is a member selected from: 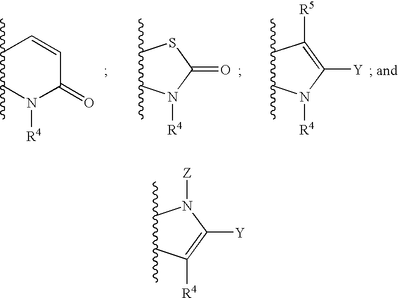
wherein
Z is substituted or unsubstituted alkyl;
Y is a member selected from H, halogen, nitro, and nitroso;
R⁴ is selected from H, substituted or unsubstituted alkyl, substituted or unsubstituted heteroalkyl, and a carrier moiety; and
R⁵ is a member selected from H, CN, OR¹², C(X¹)OR¹², C(X¹)NR¹³R¹⁴, NR¹⁵R¹⁶, SR¹², NO, halogen, substituted or unsubstituted C₁-C₆ alkyl and substituted or unsubstituted C₁-C₆ heteroalkyl
wherein
R¹² is a member selected from H, substituted or unsubstituted C₁-C₆ alkyl, substituted or unsubstituted C₁-C₆ heteroalkyl and C(O)R¹⁷
wherein
R¹⁷ is substituted or unsubstituted C₁-C₆ alkyl and substituted or unsubstituted C₁-C₆ heteroalkyl;
X¹ is a member selected from (═O), (═NH) and (═S);
R¹³ and R¹⁴ are members independently selected from H, substituted or unsubstituted C₁-C₆ alkyl and substituted or unsubstituted C₁-C₆ heteroalkyl; and
R¹⁵ and R¹⁶ are members independently selected from H, O, substituted or unsubstituted C₁-C₆ alkyl and substituted or unsubstituted C₁-C₆ heteroalkyl, or taken together, form C(O)R¹⁸;
wherein
R¹⁸ is a member selected from substituted or unsubstituted C₁-C₆ alkyl and substituted or unsubstituted C₁-C₆ heteroalkyl.

2. The compound according to claim 1, wherein R⁴ is a member selected from alkyl substituted with at least one hydroxyl moiety and heteroalkyl substituted with at least one hydroxyl moiety.

3. The compound according to claim 1, wherein R⁴ is a member selected from: 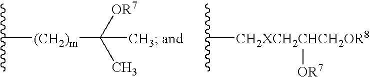
wherein
m is an integer from 1 to 10;
R⁷ and R⁸ are members independently selected from H and carrier moieties;
X is a member selected from O, S and NR⁶
wherein
R⁶ is a member selected from H, substituted or unsubstituted alkyl, substituted or unsubstituted heteroalkyl, substituted or unsubstituted aryl, substituted or unsubstituted heteroaryl and substituted or unsubstituted heterocycloalkyl.

4. The compound according to claim 1, wherein said carrier moiety is a polymer.

5. The compound according to claim 1, wherein said carrier moiety is essentially non-antigenic in a mammalian subject.

6. The compound according to claim 1, wherein said carrier moiety is a biomolecule.

7. The compound according to claim 6, wherein said carrier moiety is a member selected from a nucleic acid, an amino acid, a peptide, a peptide-amino acid, a saccharide, an antibody, an antigen, a lectin and combinations thereof.

8. The compound according to claim 2, wherein R⁴ is a saccharyl moiety.

9. The compound according to claim 8, wherein said saccharyl moiety is a member selected from substituted or unsubstituted ribofuranose and substituted or unsubstituted deoxyribofuranose.

10. The compound according to claim 9, wherein said saccharyl moiety is part of a complex, said complex comprising a member selected from a nucleic acid and a peptide-amino acid.

11. The compound according to claim 1, wherein at least one of R¹, R², R³, R⁴ and R⁵ comprise a phosphoramidite moiety.

12. The compound according to claim 11, wherein said phosphoramidite moiety has the formula:

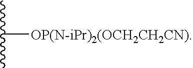

13. A pharmaceutical composition comprising a compound having the formula: 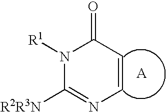
wherein
R¹, R² and R³ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl; and
ring system A is a member selected from: 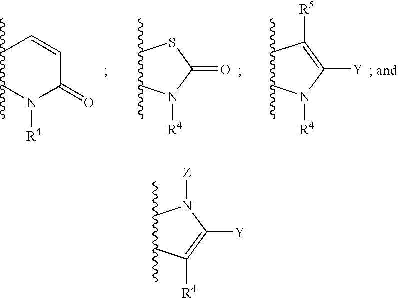
wherein
Z is substituted or unsubstituted alkyl;
Y is a member selected from H, halogen, nitro, and nitroso;
R⁴ is selected from H, substituted or unsubstituted alkyl, substituted or unsubstituted heteroalkyl and a carrier moiety; and
R⁵ is a member selected from H, CN, OR¹², C(X¹)OR¹², C(X¹)NR¹³R¹⁴, NR¹⁵R¹⁶, SR¹², NO, halogen, substituted or unsubstituted C₁-C₆ alkyl and substituted or unsubstituted C₁-C₆ heteroalkyl
wherein
R¹² is a member selected from H, substituted or unsubstituted C₁-C₆ alkyl, substituted or unsubstituted C₁-C₆ heteroalkyl and C(O)R¹⁷
wherein
R¹⁷ is substituted or unsubstituted C₁-C₆ alkyl and substituted or unsubstituted C₁-C₆ heteroalkyl;
X¹ is a member selected from (═O), (═NH) and (═S);
R¹³ and R¹⁴ are members independently selected from H, substituted or unsubstituted C₁-C₆ alkyl and substituted or unsubstituted C₁-C₆ heteroalkyl; and
R¹⁵ and R¹⁶ are members independently selected from H, O, substituted or unsubstituted C₁-C₆ alkyl and substituted or unsubstituted C₁-C₆ heteroalkyl, or taken together, form C(O)R¹⁸
wherein
R¹⁸ is a member selected from substituted or unsubstituted C₁-C₆ alkyl and substituted or unsubstituted C₁-C₆ heteroalkyl; and
a pharmaceutically acceptable carrier.

14. A nucleic acid having a sequence comprising at least one moiety having the formula: 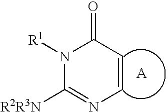
wherein
R¹, R² and R³ members independently from H, substituted or unsubstituted alkyl, substituted or unsubstituted heteroalkyl, substituted or unsubstituted aryl, substituted or unsubstituted heteroaryl and substituted or unsubstituted heterocycloalkyl; and
ring system A is a member selected from: 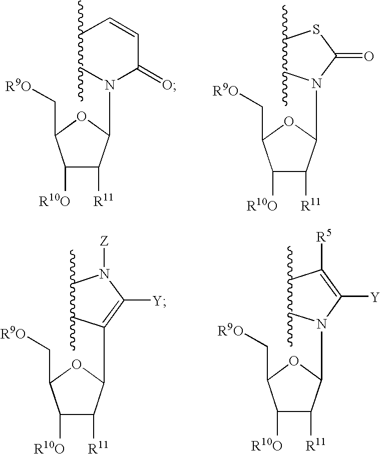
wherein
Z is substituted or unsubstituted alkyl;
Y is a member selected from H, halogen, nitro, and nitroso;
R⁵ is a member selected from H, CN, OR¹², C(X¹)OR¹², C(X¹)NR¹³R¹⁴, NR¹⁵R¹⁶, SR¹², NO, halogen, substituted or unsubstituted C₁-C₆ alkyl and substituted or unsubstituted C₁-C₆ heteroalkyl
wherein
R¹² is a member selected from H, substituted or unsubstituted C₁-C₆ alkyl, substituted or unsubstituted C₁-C₆ heteroalkyl and C(O)R¹⁷
wherein
R¹⁷ is substituted or unsubstituted C₁-C₆ alkyl and substituted or unsubstituted C₁-C₆ heteroalkyl;
X¹ is a member selected from (═O), (═NH) and (═S);
R¹³ and R¹⁴ are members independently selected from H, substituted or unsubstituted C₁-C₆ alkyl and substituted or unsubstituted C₁-C₆ heteroalkyl; and
R¹⁵ and R¹⁶ are members independently selected from H, O, substituted or unsubstituted C₁-C₆ alkyl and substituted or unsubstituted C₁-C₆ heteroalkyl, or taken together, form C(O)R¹⁸
wherein
R¹⁸ is a member selected from substituted or unsubstituted C₁-C₆ alkyl and substituted or unsubstituted C₁-C₆ heteroalkyl;
R⁹ and R¹⁰ are members independently selected from H, and a nucleic acid; and
R¹¹ is a member selected from H, OH, and a nucleic acid.

15. The nucleic acid sequence according to claim 14, having a CpG format.

16. A pharmaceutical composition comprising a nucleic acid having a sequence comprising at least one moiety having the formula: 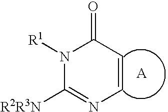
wherein
R¹, R² and R³ are members independently from H, substituted or unsubstituted alkyl, substituted or unsubstituted heteroalkyl, substituted or unsubstituted aryl, substituted or unsubstituted heteroaryl and substituted or unsubstituted heterocycloalkyl; and
ring system A is a member selected from: 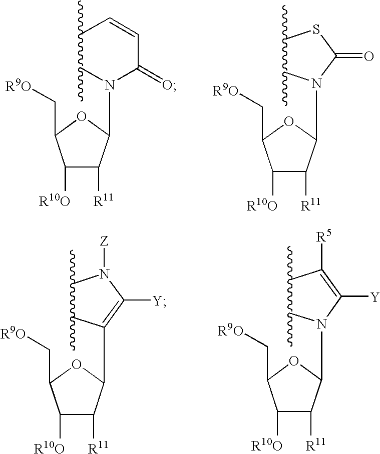
wherein
Z is substituted or unsubstituted alkyl;
Y is a member selected from H, halogen, nitro, and nitroso;
R⁵ is a member selected from H, CN, OR¹², C(X¹)OR¹², C(X¹)NR¹³R¹⁴, NR¹⁵R¹⁶, SR¹², NO, halogen, substituted or unsubstituted C₁-C₆ alkyl and substituted or unsubstituted C₁-C₆ heteroalkyl
wherein
R¹² is a member selected from H, substituted or unsubstituted C₁-C₆ alkyl, substituted or unsubstituted C₁-C₆ heteroalkyl and C(O)R¹⁷
wherein
R¹⁷ is substituted or unsubstituted C₁-C₆ alkyl and substituted or unsubstituted C₁-C₆ heteroalkyl;
X¹ is a member selected from (═O), (═NH) and (═S);
R¹³ and R¹⁴ are members independently selected from H, substituted or unsubstituted C₁-C₆ alkyl and substituted or unsubstituted C₁-C₆ heteroalkyl; and
R¹⁵ and R¹⁶ are members independently selected from H, O, substituted or unsubstituted C₁-C₆ alkyl and substituted or unsubstituted C₁-C₆ heteroalkyl, or taken together, form C(O)R¹⁸
wherein
R¹⁸ is a member selected from substituted or unsubstituted C₁-C₆ alkyl and substituted or unsubstituted C₁-C₆ heteroalkyl;
R⁹ and R¹⁰ are members independently selected from H, and a nucleic acid;
R¹¹ is a member selected from H, OH and a nucleic acid; and a pharmaceutically acceptable carrier.

17. A method of activating an immune system in a mammal in need of such activation, the method comprising administering to the mammal a therapeutically effective amount of a pharmaceutical composition, wherein the pharmaceutical composition comprises a nucleic acid of claim 14, wherein the nucleic acid comprises a toll-like receptor (TLR) ligand.

18. The method of claim 17, wherein the TLR ligand binds to a TLR expressed on an endosomal membrane.

19. The method of claim 17, wherein the composition further comprises a CpG oligonucleotide (ISS-ODN).

20. The method of claim 17, further comprising administration of an IMPDH inhibitor.

21. The method of claim 17, wherein the composition is administered to a mucus membrane.

22. The method of claim 17, wherein said TLR ligand is a homofunctional TLR ligand polymer.

23. The method of claim 22, wherein the homofunctional TLR ligand polymer comprises a TLR ligand selected from the group consisting of a TLR-7 ligand and a TLR-8 ligand.

24. The method of claim 23, wherein the homofunctional TLR ligand polymer comprises a TLR-7 ligand.

25. The method of claim 24, wherein the TLR-7 ligand is a member selected from the group consisting of a 7-thia-8-oxoguanosinyl (TOG) moiety, a 7-deazaguanosinyl (7DG) moiety, and an imiquimod moiety.

26. The method of claim 23, wherein the homofunctional TLR ligand polymer comprises a TLR-8 ligand.

27. The method of claim 26, wherein the TLR-8 ligand is a resiquimod moiety.

28. The method of claim 17, wherein said TLR ligand is a heterofunctional TLR ligand polymer.

29. The method of claim 28, wherein said heterofunctional TLR ligand polymer comprises a TLR-7 ligand and a member selected from the group consisting of a TLR-8 ligand and a TLR-9 ligand.

30. The method of claim 28, wherein said heterofunctional TLR ligand polymer comprises a TLR-7 ligand, a TLR-8 ligand, and a TLR-9 ligand.

31. The method of claim 28, wherein said heterofunctional TLR ligand polymer comprises a TLR-8 ligand and a TLR-9 ligand.

32. A method of enhancing resistance to infection in a mammal in need of such enhancement of resistance, the method comprising administering to the mammal a therapeutically effective amount of a pharmaceutical composition, wherein the pharmaceutical composition comprises a nucleic acid of claim 14, wherein the nucleic acid comprises a toll-like receptor (TLR) ligand.

33. The method of claim 32, wherein the TLR ligand binds to a TLR expressed on an endosomal membrane.

34. The method of claim 32, wherein the composition further comprises a CpG oligonucleotide (ISS-ODN).

35. The method of claim 32, further comprising administration of an IMPDH inhibitor.

36. The method of claim 32, wherein the composition is administered to a mucus membrane.

37. The method of claim 32, wherein said TLR ligand is a homofunctional TLR ligand polymer.

38. The method of claim 37, wherein the homofunctional TLR ligand polymer comprises a TLR ligand selected from the group consisting of a TLR-7 ligand and a TLR-8 ligand.

39. The method of claim 38, wherein said homofunctional TLR ligand polymer comprises a TLR-7 ligand.

40. The method of claim 39, wherein said TLR-7 ligand is a member selected from the group consisting of a 7-thia-8-oxoguanosinyl (TOG) moiety, a 7-deazaguanosinyl (7DG) moiety, and an imiquimod moiety.

41. The method of claim 38, wherein the homofunctional TLR ligand polymer comprises a TLR-8 ligand.

42. The method of claim 41, wherein the TLR-8 ligand is a resiquimod moiety.

43. The method of claim 32, wherein said TLR ligand is a heterofunctional TLR ligand polymer.

44. The method of claim 43, wherein said heterofunctional TLR ligand polymer comprises a TLR-7 ligand and a member selected from the group consisting of a TLR-8 ligand and a TLR-9 ligand.

45. The method of claim 43, wherein said heterofunctional TLR ligand polymer comprises a TLR-7 ligand, a TLR-8 ligand, and a TLR-9 ligand.

46. The method of claim 43, wherein said heterofunctional TLR ligand polymer comprises a TLR-8 ligand and a TLR-9 ligand.

47. The method of claim 32, wherein the infection is caused by a virus.

48. The method of claim 47, wherein the virus is an interferon-sensitive virus.

49. The method of claim 32, wherein the infection is caused by a bacteria.

50. The method of claim 49, wherein the bacteria causes an intracellular bacterial infection.

51. The method of claim 49, wherein an antibiotic is also administered to the mammal.

52. A method of treating a viral infection in a mammal in need of such treatment, the method comprising administering a TLR ligand, and
administering an IMPDH inhibitor.

53. The method of claim 52, wherein the IMPDH inhibitor is mizoribine, an entiomer of mizoribine, mizoribine base, mizoribine aglycone, or a prodrug of such compound.

54. The method of claim 52, wherein the viral infection is caused by an RNA virus.

55. The method of claim 54, further comprising administering a synthetic TLR ligand.

56. The method of claim 54, wherein the viral infection is caused by an RNA virus selected from the group consisting of a coronavirus that causes Severe Acute Respiratory Syndrome (SARS) and a Hepatitis C Virus.

57. The method of claim 54, wherein the RNA virus is mutated and does not cause an induction of interferon synthesis.

58. The method of claim 54, wherein the IMPDH inhibitor is administered directly to the site of viral infection.

59. The method of claim 58, wherein the RNA virus is a coronavirus that causes SARS and the IMPDH inhibitor is administered to a lung.

60. The method of claim 52, wherein the viral infection is caused by a DNA virus.

61. The method of claim 60, wherein the TLR ligand is a synthetic TLR ligand.

62. The method of claim 60, wherein the DNA virus is a Hepatitis B virus.

63. The method of claim 60, wherein the IMPDH inhibitor is given systemically.

64. A method for treating cancer comprising administering to a subject in need of such treatment a therapeutically effective amount of
(a) a member selected from an inhibitor of inosine monophosphate dehydrogenase (IMPDH), an enantiomer of such a compound, a prodrug of such a compound, a pharmaceutically acceptable salt of such a compound, and combinations thereof; and
(b) an interferon inducer.

65. The method of claim 64, wherein the cancer is an interferon-sensitive cancer.

66. The method of claim 65, wherein the interferon-sensitive cancer is a member selected from a leukemia, a lymphoma, a myeloma, a melanoma, and a renal cancer.

67. The method of claim 64, wherein the IMPDH inhibitor is selected from the group consisting of mizoribine, mizoribine base, mizoribine aglycone, mycophenolic acid, mycophenolate mofetil, Tiazofurin and ribavirin.

68. The method of claim 64, further comprising administration of therapeutically effective amount of a Type I interferon.

69. The method of claim 64, wherein the interferon inducer comprises a therapeutically effective amount of a pharmaceutical composition, wherein the pharmaceutical composition comprises a nucleic acid of claim 14, wherein the nucleic acid comprises a toll-like receptor (TLR) ligand.

70. The method of claim 69, wherein the TLR ligand binds to a TLR expressed on an endosomal membrane.

71. The method of claim 69, wherein the composition further comprises a CpG oligonucleotide (ISS-ODN).

72. The method of claim 69, wherein the composition is administered to a mucus membrane.

73. The method of claim 69, wherein the TLR ligand is a homofunctional TLR ligand polymer.

74. The method of claim 73, wherein the homofunctional TLR ligand polymer comprises a TLR ligand selected from the group consisting of a TLR-7 ligand and a TLR-8 ligand.

75. The method of claim 74, wherein said homofunctional TLR ligand polymer comprises a TLR-7 ligand.

76. The method of claim 75, wherein said TLR-7 ligand is a member selected from the group consisting of a 7-thia-8-oxoguanosinyl (TOG) moiety, a 7-deazaguanosinyl (7DG) moiety, and an imiquimod moiety.

77. The method of claim 74, wherein the homofunctional TLR ligand polymer comprises a TLR-8 ligand.

78. The method of claim 77, wherein the TLR-8 ligand is a resiquimod moiety.

79. The method of claim 69, wherein said TLR ligand is a heterofunctional TLR ligand polymer.

80. The method of claim 79, wherein said heterofunctional TLR ligand polymer comprises a TLR-7 ligand and a member selected from the group consisting of a TLR-8 ligand and a TLR-9 ligand.

81. The method of claim 79, wherein said heterofunctional TLR ligand polymer comprises a TLR-7 ligand, a TLR-8 ligand, and a TLR-9 ligand.

82. The method of claim 79, wherein said heterofunctional TLR ligand polymer comprises a TLR-8 ligand and a TLR-9 ligand.

83. A method for treating an autoimmune disease comprising administering to a subject in need of such treatment a therapeutically effective amount of
(a) a member selected from an inhibitor of inosine monophosphate dehydrogenase (IMPDH), an enantiomer of such a compound, a prodrug of such a compound, a pharmaceutically acceptable salt of such a compound, and combinations thereof; and
(b) an interferon inducer.

84. The method of claim 83, wherein the IMPDH inhibitor is selected from the group consisting of mizoribine, mizoribine base, mizoribine aglycone, mycophenolic acid, mycophenolate mofetil, Tiazofurin and ribavirin.

85. The method of claim 83, wherein the autoimmune disease is multiple sclerosis.

86. The method of claim 83, further comprising administering a therapeutically effective amount of a Type I interferon.

87. The method of claim 83, wherein the interferon inducer comprises a therapeutically effective amount of a pharmaceutical composition, wherein the pharmaceutical composition comprises a nucleic acid of claim 14, wherein the nucleic acid comprises a toll-like receptor (TLR) ligand.

88. The method of claim 87, wherein the TLR ligand binds to a TLR expressed on an endosomal membrane.

89. The method of claim 87, wherein the composition further comprises a CpG oligonucleotide (ISS-ODN).

90. The method of claim 87, wherein the composition is administered to a mucus membrane.

91. The method of claim 87, wherein said TLR ligand is a homofunctional TLR ligand polymer.

92. The method of claim 91, wherein the homofunctional TLR ligand polymer comprises a TLR ligand selected from the group consisting of a TLR-7 ligand and a TLR-8 ligand.

93. The method of claim 92, wherein said homofunctional TLR ligand polymer comprises a TLR-7 ligand.

94. The method of claim 93, wherein said TLR-7 ligand is a member selected from the group consisting of a 7-thia-8-oxoguanosinyl (TOG) moiety, a 7-deazaguanosinyl (7DG) moiety, and an imiquimod moiety.

95. The method of claim 92, wherein the homofunctional TLR ligand polymer comprises a TLR-8 ligand.

96. The method of claim 95, wherein the TLR-8 ligand is a resiquimod moiety.

97. The method of claim 87, wherein said TLR ligand is a heterofunctional TLR ligand polymer.

98. The method of claim 97, wherein said heterofunctional TLR ligand polymer comprises a TLR-7 ligand and a member selected from the group consisting of a TLR-8 ligand and a TLR-9 ligand.

99. The method of claim 97, wherein said heterofunctional TLR ligand polymer comprises a TLR-7 ligand, a TLR-8 ligand, and a TLR-9 ligand.

100. The method of claim 97, wherein said heterofunctional TLR ligand polymer comprises a TLR-8 ligand and a TLR-9 ligand.

101. A method of treating a disease accessible to topical treatment in a subject in need of such treatment comprising administering a therapeutically effective amount of an interferon inducer, wherein said interferon inducer is given topically or delivered directly to a diseased tissue; and
administering a therapeutically effective amount of a member selected from an inhibitor of inosine monophosphate dehydrogenase (IMPDH), an enantiomer of such a compound, a prodrug of such a compound, a pharmaceutically acceptable salt of such a compound, and combinations thereof.

102. The method of claim 101, wherein the interferon inducer is a TLR ligand.

103. The method of claim 102, wherein the TLR ligand is selected from the group consisting of resiquimod, imiquimod, and ISS-ODN.

104. The method of claim 102, wherein the TLR ligand is a nucleic acid of claim 14.

105. The method of claim 101, wherein the IMPDH inhibitor is administered systemically.

106. The method of claim 101, wherein the IMPDH inhibitor is a member selected from the group consisting of mizoribine, mizoribine base, and mizoribine aglycone.

107. The method of claim 101, wherein the disease accessible to topical treatment is selected from the group consisting of cancer and precancerous conditions.

108. The method of claim 107, wherein the cancer is selected from the group consisting of melanoma, superficial bladder cancer, actinic keratoses, intraepithelial neoplasia, and basal cell skin carcinoma.

109. The method of claim 107, wherein the precancerous condition is selected from the group consisting of actinic keratoses and intraepithelial neoplasia.

110. The method of claim 101, wherein the disease accessible to topical treatment is a viral disease.

111. The method of claim 110, wherein the viral disease is a selected from the group consisting of a human papilloma virus infection, a molluscum contagiosum, and a herpes virus infection.

112. A method of treating cancer in a subject in need of such treatment comprising administering a therapeutically effective amount of a member selected from mizoribine, mizoribine base, mizoribine aglycone, an enantiomer of such a compound, a prodrug of such a compound, a pharmaceutically acceptable salt of such a compound, and combinations thereof; in combination with a therapeutically effective amount of Type I interferon.

113. The method of claim 112, wherein the cancer is a member selected from a leukemia, a lymphoma, a myeloma, a melanoma, and a renal cancer.

114. A method of treating a viral infection in a subject in need of such treatment comprising administering a therapeutically effective amount of a member selected from mizoribine, mizoribine base, mizoribine aglycone, an enantiomer of such a compound, a prodrug of such a compound, a pharmaceutically acceptable salt of such a compound, and combinations thereof; in combination with a therapeutically effective amount of Type I interferon.

115. The method of claim 114, wherein the viral infection is caused by a virus selected from the group consisting of a coronavirus that causes Severe Acute Respiratory Syndrome (SARS), a Hepatitis B virus, and a Hepatitis C Virus.

116. A method of treating an autoimmune disease in a subject in need of such treatment comprising administering a therapeutically effective amount of a member selected from mizoribine, mizoribine base, mizoribine aglycone, an enantiomer of such a compound, a prodrug of such a compound, a pharmaceutically acceptable salt of such a compound, and combinations thereof; in combination with a therapeutically effective amount of Type I interferon.

117. The method of claim 116, wherein the autoimmune disease is Multiple Sclerosis.

118. A method of treating Crohn's Disease in a subject in need of such treatment comprising administering a member selected from an inhibitor of inosine monophosphate dehydrogenase (IMPDH), an enantiomer of such a compound, a prodrug of such a compound, a pharmaceutically acceptable salt of such a compound, and combinations thereof, and a member selected from the group consisting of probiotics and glycolipids.
